(19) United States
(12) Patent Application Publication
(10) Pub. No.: US 2024/0428934 A1
(43) Pub. Date: Dec. 26, 2024

(54) METHODS AND SYSTEMS FOR PRESENTATION OF HISTORICAL MULTI-CHANNEL SERVICE PLATFORMS

(71) Applicant: Polyview Health, Inc., San Diego, CA (US)

(72) Inventors: Jason Hwang, Henderson, NV (US); Gregory Deocampo, Safety Harbor, FL (US); Dimitri Arges, Henderson, NV (US)

(21) Appl. No.: 18/746,384

(22) Filed: Jun. 18, 2024

Related U.S. Application Data
(60) Provisional application No. 63/509,910, filed on Jun. 23, 2023.
(60) Provisional application No. 63/509,973, filed on Jun. 23, 2023.
(60) Provisional application No. 63/510,006, filed on Jun. 23, 2023.
(60) Provisional application No. 63/510,019, filed on Jun. 23, 2023.
(60) Provisional application No. 63/510,608, filed on Jun. 27, 2023.
(60) Provisional application No. 63/604,930, filed on Dec. 1, 2023.

(51) Int. Cl. G16H 40/67; G16H 80/00; H04L 12/18

(52) U.S. Cl. CPC G16H 40/67; G16H 80/00; H04L 12/1831

(57) ABSTRACT

Methods and systems are described herein for presenting historical multi-channel service platform sessions. The method comprises receiving a request to establish a communication session between one or more user devices. A first user device may be associated with a healthcare provider and a second user device may be associated with a patient. The method may further comprise receiving user profile data, wherein the user profile data pertains to at least one of the one or more user devices of the communication session. One or more post-session environments may be generated that are associated with the one or more user devices. The one or more post-session environments are configured to allow the one or more user devices to evaluate content associated with the communication session after the virtual communication session concludes. A communication may be transmitted to the one or more user devices containing a link to a post-session environment.

### CROSS-REFERENCE TO RELATED APPLICATIONS

The present patent application claims the benefit of priority to U.S. Provisional Patent Applications 63/509,910, 63/509,973, 63/510,006, and 63/510,019, all of which were filed Jun. 23, 2023; U.S. Provisional Patent Application 63/510,608, filed Jun. 27, 2023; and U.S. Provisional Patent Application 63/604,930, filed Dec. 1, 2023, which are all incorporated herein by reference in their entirety for all purposes.

### TECHNICAL FIELD

This disclosure relates generally to communication session environments. More specifically, this disclosure relates to generating a communication session review environment customized according to one or more user devices on a multi-channel service platform.

### BACKGROUND

Telehealth can be conducted between a patient and a healthcare provider over a variety of communication channels. These telehealth calls are often facilitated in a standard format or based on third-party's pre-existing systems (e.g., Zoom, Microsoft Teams, Facetime, etc.). Because of the basic nature of current telehealth calls, the calls themselves may not be as beneficial or as efficient as they could be for both the patient and the healthcare provider. For example, it might be more difficult for a patient or healthcare provider to focus on the telehealth call or provide relevant information. As another example, telehealth calls often cause the patient to feel disengaged from the appointment and the healthcare provider, thus creating an environment where the patient might not trust the healthcare provider's advice or might not feel comfortable asking questions.

### SUMMARY

Methods are described herein for provisioning post-session environments associated with communication sessions. For example, a computer-implemented method may include receiving a request, via a communication system, to generate a video segment associated with a communication session between one or more user devices, wherein a first device of the one or more user devices of the communication session is a healthcare provider; receiving user profile data, wherein the user profile data pertains to at least one of the one or more user devices of the communication session; generating one or more post-session environments associated with the one or more user devices, wherein the one or more post-session environments are configured to present a modified representation of the communication session after the communication session terminates; and transmitting a first communication to the first device including a link to a first post-session environment, and wherein the first post-session environment is distinct from other second post-session environments of the one or more post-session environments.

Systems are described herein for presenting historical multi-channel service platform sessions. The systems include one or more processors and a non-transitory computer-readable storage medium storing instructions that, when executing by the one or more processors, cause the one or more processors to perform any of the methods as previously described.

A non-transitory computer-readable medium described herein may store instructions which, when executed by one or more processors, cause the one or more processors to perform any of the methods as previously described.

These illustrative examples are mentioned not to limit or define the disclosure, but to aid understanding thereof. Additional embodiments are discussed in the Detailed Description, and further description is provided there.

### BRIEF DESCRIPTION OF THE DRAWINGS

Features, instances, and advantages of the present disclosure are better understood when the following Detailed Description is read with reference to the accompanying drawings.

FIG. 1 illustrates a block diagram of example communication network configured to facilitate secured communication sessions between users according to aspects of the present disclosure.

FIG. 2A illustrates an example patient user interface of the communication network configured for a particular communication session according to aspects of the present disclosure.

FIG. 2B illustrates an example provider user interface of the communication network configured for a particular communication session according to aspects of the present disclosure.

FIG. 2C illustrates an example temporary environment user interface of the communication network configured for a particular user device according to aspects of the present disclosure.

FIG. 3A illustrates an example post-session environment of the communication network configured for a particular user device according to aspects of the present disclosure.

FIG. 3B illustrates an example post-session environment of the communication network configured for a particular user device according to aspects of the present disclosure.

FIG. 4 illustrates a flowchart of an example process for generating a review environment of the communication network configured for a particular user device according to aspects of the present disclosure.

FIG. 5 illustrates an example computing device according to aspects of the present disclosure.

### DETAILED DESCRIPTION

Various instances of the disclosure are discussed in detail below. While specific implementations are discussed, it should be understood that this is done for illustration purposes only. A person skilled in the relevant art will recognize that other components and configurations may be used without parting from the spirit and scope of the disclosure.

Methods and systems are described herein for providing a communication session review environment within a multi-channel service platform. In some instances, a communication session may be conducted between a first user device associated with a patient and a second user device associated with a healthcare provider as part of a telehealth session. The healthcare provider may be a doctor, nurse, therapist, and/or other healthcare professional. One or more other user devices may also be connected to the communication session. The one or more other user devices may be associated with other users (e.g., such as additional patients, users associated with the patient such as a nurse, additional healthcare providers, etc.). For example, an adult child of an elderly parent may participate in a communication session with the elderly parent and a healthcare provider of the elderly parent.

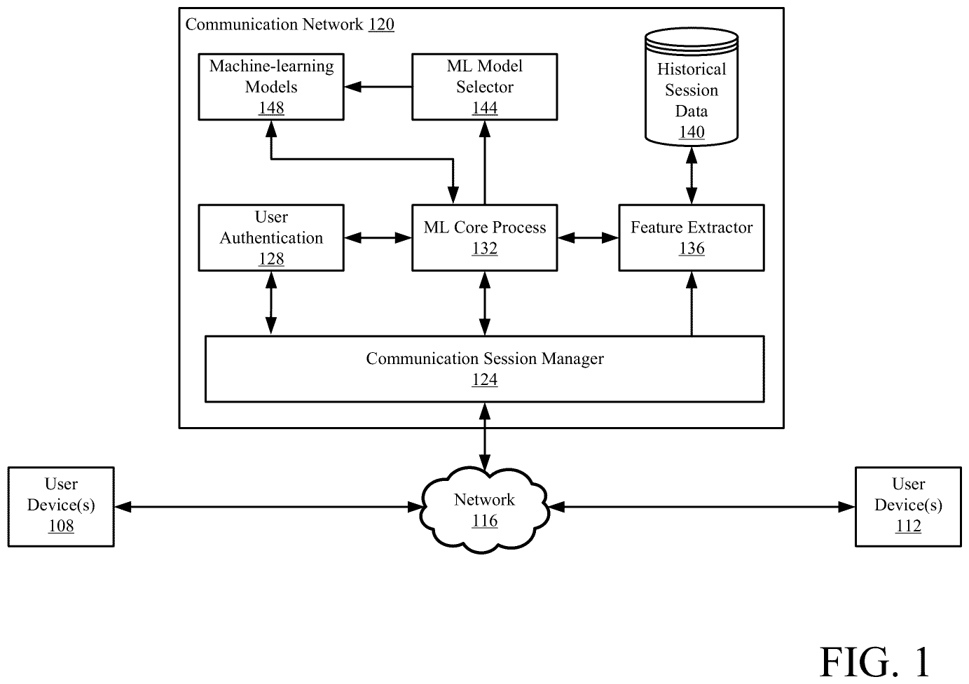

The first user device may transmit a communication session request to a communication network (e.g., communication network 120 of FIG. 1, an application, network, etc.). The communication session may be facilitated over one or more communication channels (e.g., telephone or other audio-based communication channels, video or other audiovisual-based communication session, text such as text messaging or instant messaging, combinations thereof, or the like. In some instances, the communication channel may be asynchronous, or communications may be transmitted over the communication channel asynchronously. For example, the first user device may communicate over an audio channel and lack a capability of receiving or processing data received over a video channel (e.g., due to permissions or other security settings, processing capabilities, available resources, available bandwidth, etc.). The second user device may communicate over both audio and video, etc. such that the first user device may receive audio from the second user device and the second user device may receive both audio and video from the first user device.

The communication session request may include a set of communication session parameters. The set of communication session parameters may include, but are not limited to, a quantity of user devices that are authorized to connect to the communication session, an identification of the user devices or the users thereof (e.g., such a device identifier, Internet Protocol address, email address, phone number, username, a user identifier, combinations thereof, or the like), a length of the communication session, an identification of one or more communication channels authorized for the communication session (e.g., such as, audio, video, audiovisual, text messaging, email, instant messaging, combinations thereof, or the like), video settings, sound settings, collaborative environment parameters, privacy and/or encryption parameters, artificial intelligence accessibility, date and time, etc. The set of communication session parameters may be modified at any time prior to and/or during the communication session.

In an illustrative example, the set of communication session parameters may include an identification of one or more user devices that are configured to connect to the communication session. The one or more user devices may include the first user device and/or the second user device. The one or more user devices may also include user devices operated by other healthcare providers, other patients, individuals associated with the patient (e.g., such as a social worker, nurse, aide, etc., a third party, etc.), combinations thereof, other the like. The first user device may identify the one or more user devices to be invite the one or more user devices to connect to the communication session.

The communication network may transmit a notification to the one or more user devices invited to the communication session using information of the set of communication session parameters. The notification may include a representation of one or more communication session parameters of the set of communication session parameters. The one or more user devices may accept or decline the communication session. A user device of the one or more user devices may request a modification to one or more of the communication session parameters (e.g., such the date/time of the communication session, identification of user devices authorized to access the communication session, etc.). The communication network may receive a response from the one or more user devices (or other user devices associated with the communication request). The one or more user devices may modify a response of accepting or declining the communication session request (e.g., changing an acceptance to a decline, changing a decline to an acceptance, etc.). The communication session parameters may determine an action if no response is received from a user device of the one or more user devices. In some instances, the communication network may indicate that a lack of response from the user device may be an acceptance. In other instances, the communication network may indicate that a lack of response from the user device may be a decline.

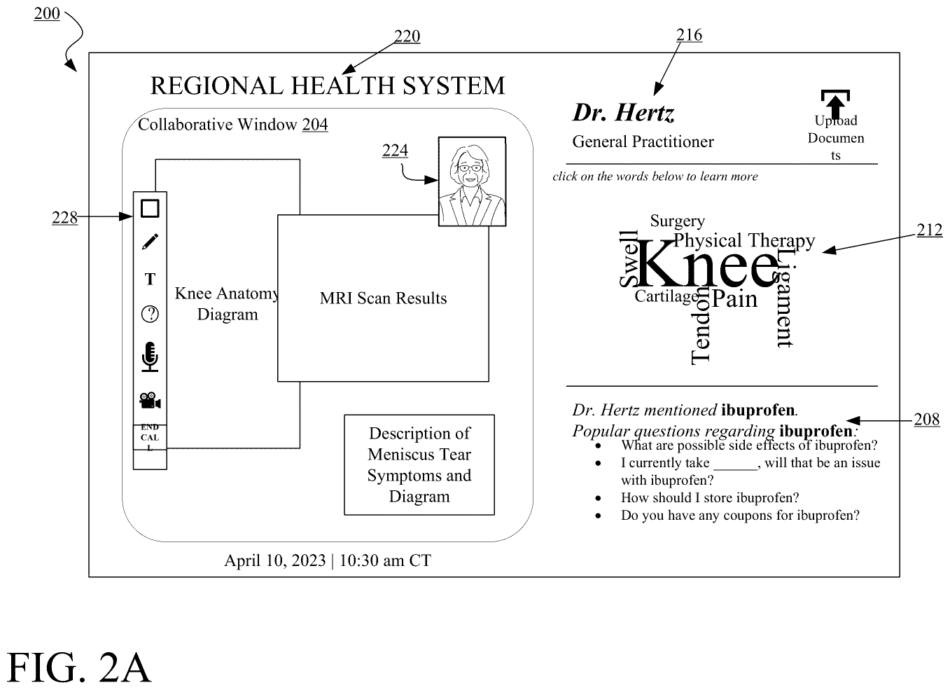

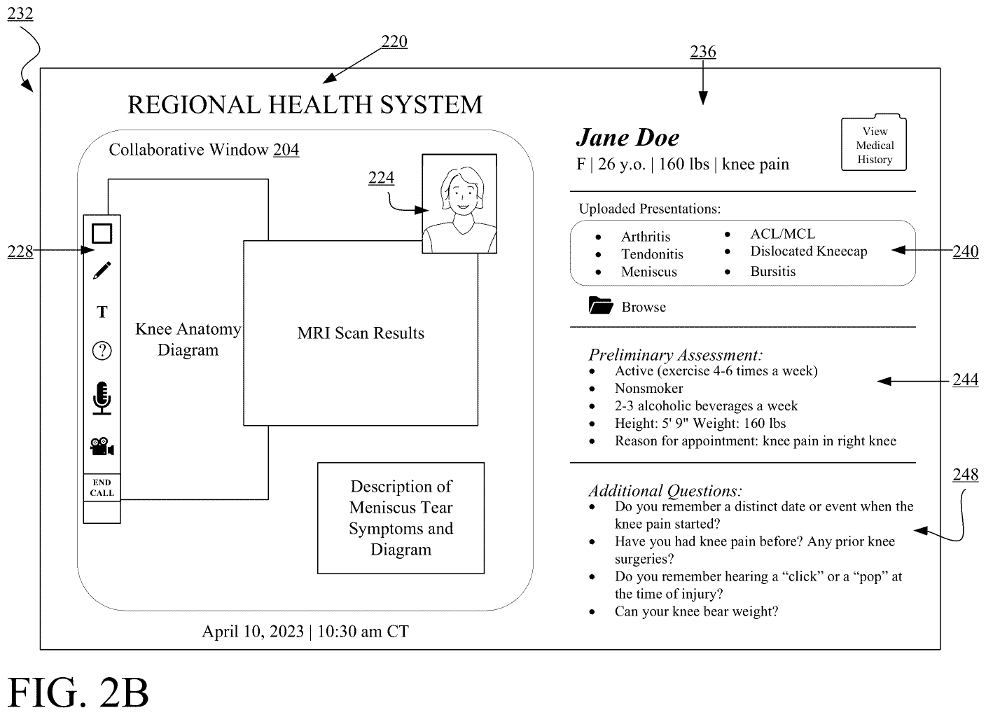

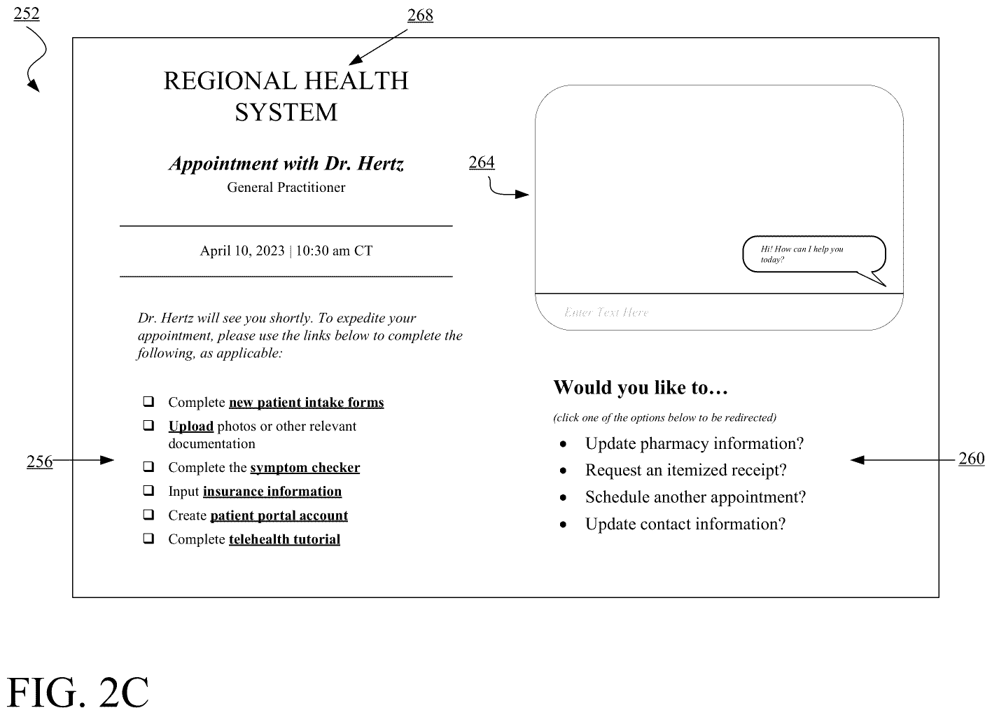

In some examples, the communication network may facilitate access to a telehealth environment (e.g., such as a virtualized environment, container, partition, etc.). The telehealth environment may be operated by the communication network, the healthcare provider, an entity associated with the healthcare provider, etc. In some instances, the telehealth environment may be an application, website, or like configured to store and provide information associated with the first user device and facilitate the communication session. In other instances, the telehealth environment may be a virtualized environment (e.g., such as, but not limited to a virtual machine, secure processing environment, or the like) configured to execute processes and/or applications, store and/or provide information associated with the user device, facilitate communications between two or more devices, etc. The telehealth environment may include a sub-environment for facilitating the communication session (e.g., as shown FIG. 2A).

The telehealth environment may include a linked post-session environment. The post-session environment may include an identification of previous communication sessions associated with the first user device. The telehealth environment may include an application or plugin configured to replay a previous communication session. In some instances, the telehealth environment may include a link to an environment associated with the healthcare provider where the previous communication session may be stored. The first user device may access the link to connect to the environment associated with the healthcare provider to access the previous communication session. Alternatively, the communication network may embed the previous communication session hosted by the environment associated with the healthcare provider within the telehealth environment to enable the first user device to access the previous communication session without leaving the telehealth environment. The post-session environment may also include resources presented by user devices during communications sessions, resources provided by user devices before or after a communication session (e.g., charts, explanations, prescriptions, test results, notes, instructions, etc.), access to artificial intelligence (AI) resources (e.g., natural language processors for speech-to-text, text-to-speech, translation, classification, etc.; large language models or other generative models for automated communication and information generation, etc.), other resources, administrative links (e.g., pay bill, schedule appointment, etc.), transcripts of prior communication sessions, potential questions for the healthcare provider, etc.

The communication network may provide access to the communication session when a current time corresponds to a time identified by a communication session parameter. The communication network may establish the communication session through the telehealth environment using a sub-environment configured to enable communications over one or more communications channels identified by a communication session parameter of the set of communication session parameters. Alternatively, the communication network may establish the communication session using a third-party environment (e.g., video conferencing application or environment provided by an entity other than the communication network, etc.). The first user device and the one or more user devices may access the communication network and/or the communication session using via the Internet, local or wide area network, a cellular network, etc.

In some examples, the communication network may include an access sequence restriction that defines an order in which user devices may access the communication session. The access sequence restriction may comprise an access sequence order, wherein the access sequence order may position the first user device and the one or more other user devices sequentially. The communication network may connect the one or more user devices positioned lower in the access sequence order to a temporary environment (e.g., a virtual waiting room, etc.) until the one or more user devices positioned higher in the access sequence order connect to the communication session. For example, the communication network may indicate that the first user device cannot join the communication session until the second user device connects to the communication session. The communication network may connect the first user device to the temporary environment until the second user device connects to the communication session.

The temporary environment may enable user devices to access information associated with the communication session and transmit information to the communication network or healthcare provider (e.g., intake healthcare forms, medical history, insurance information, etc.). The information associated with the communication session can include a tutorial or other introductory information that provides information on features of the communication network and communication session as well as how to access the features, information associated with the healthcare provider (e.g., such as names and/or address of healthcare practitioners, services offered by the healthcare provider, branches of medicine practiced by the healthcare provider, affiliated healthcare providers, emergency information, etc.).

The temporary environment may include one or more automated services (e.g., chatbots, large language models, generative models, natural language understanding models, image and/or audio processors, etc.) configured to interact with the user devices of the temporary environment over various communication channels. The one or more automated services may be configured to communicate using natural language communications in a language selected by a user device communicating with the automated services. The user devices may ask questions related to the communication session or the purpose for the communication session, request information associated with the healthcare provider or communication network, etc. and receive responses from the automated service in a natural language format and in the select language. In some instances, the second user device may be presented with the questions or requests for information and provide the response in place of or in addition to the automated service. The communication network may mark responses generated by an automated service different from responses generated by the healthcare provider to enable a user to determine an origin of a particular response.

In some instances, the resources available in the temporary environment are determined by an associated health system. The associated health system may be a system affiliated with the clinic, office, hospital, etc. where the healthcare provider conducts healthcare services to patients. For example, the resources available in the temporary environment may be a map of the hospital, operating hours of the clinic and/or hospital, a link to a webpage associated with the associated health system, or any other resource that may be specific to the associated health system. The healthcare provider may also select the resources available in the temporary environment. As another example, if the healthcare provider receives information indicating that the patient has a sore throat, the healthcare provider may generate a symptom checker for the patient to complete within the temporary environment, provide diagnostic information associated with sore throats and possible causes, provide treatment information, etc.

The communication session may be facilitated by a communication session environment within the telehealth environment. The communication session environment may include a user interface generated by the communication network to facilitate the communication session. The user interface may be different for each user device or class of user devices connected to the communication session. For example, user devices associated with healthcare providers may be presented with a different user interface than user devices associated with patients. The user interface presented to the first user device may appear similar to the user interface illustrated in FIG. 2A, while the user interface presented to the second user device may appear similar to the user interface illustrated in FIG. 2B. The user interface may enable presentation of information associated with the communication session during the communication session. The communication network may enable any user device connected to the communication session to share content (e.g., read and/or write to the user interface, etc.) based upon the communication session parameters. For example, the communication session parameters may enable the second user device to share content and/or read/write to the user interface while the first user device may only display information provided by the healthcare provider.

The user interface may display resources provided by user devices connected to the communication session and/or resources provided by one or more machine-learning models. For example, the communication network may utilize a natural language processing (“NLP”) machine-learning model to present a live transcript of the communication session to a user device operated by a user that may be hearing impaired. The resources may be determined by preferences of the associated health system and/or preferences of the first user device. The resources provided through the user interface may also be determined by the second user device. For example, the second user device connected to the communication session may limit access to an AI-assisted question generator from the user interface of the first user device until the last ten minutes of the communication session.

In some examples, the communication network may provide user devices associated with healthcare providers access to additional resources pertaining to healthcare services. For example, the healthcare provider may have access to patient health records, test results, presentations associated with the communication session or the first user device (e.g., charts, graphs, images, videos, etc.). The first user device may have access to other resources. For example, the first user device may be presented with a set of pre-generated questions generated by a machine-learning model (e.g., such an NLP machine-learning model, large language model, etc.) or pre-generated based on an association with an issue for which the communication session was requested.

Within the communication session environment for the first user device and the one or more second user devices, there may be a collaborative window synchronized amongst all user devices. The collaborative window may be displayed by the user interface of a user device. The collaborative window may be updated in real-time and may enable any user device connected to the communication session to edit the collaborative window according to the collaborative session parameters. The edits may be shared in real-time to user devices connected to the communication session. The collaborative window may be associated with one or more settings configured within the communication session parameters. For example, the communication session parameters may grant permissions to one or more user devices permitted to edit/share content on the collaborative window. The communication session parameters may also determine what types of content may be shared on the collaborative window (e.g., videos, photos, diagrams, presentations, text, etc.). The collaborative window may contain a dynamic video view, wherein the video feed of a user's face from the user device currently outputting speech or sound data may be transmitted to the user devices connected to the communication session via the collaborative window.

Over the duration of the communication session, the communication network may utilize the communication (e.g., audio, visual, etc.) between the user devices connected to the communication session and generate a data package containing a recording of the communication session (e.g., containing audio, visual, etc. content outputted over the duration of the communication session) and data generated by other AI-assisted resources. After the communication session is concluded, the user devices connected to the communication session may receive a notification (e.g., via email, telehealth environment notification, text message, etc.) indicating the data package is available for examination. The data package accessible to each user device may differ based on the user device. For example, the data package of each user device may present a representation of the communication session that was presented to that device during the communication session. The first user device may access a data package with a representation of the communication session that was presented to the first user during the communication session. In some instances, the data package includes additional data derived from the communication session such as additional information about objects presented in the user interface (including the collaborative environment, etc.), statements made by the healthcare provider or patient, etc. The additional data may be generated automatically (e.g., by one or more of the AI resources or automated services previously described), generated by the healthcare provider, generated by the patient, or another entity that was connected to the communication session.

FIG. 1 illustrates a block diagram of example communication network configured to facilitate secured communication sessions between users according to aspects of the present disclosure. Communication network 120 may facilitate communication sessions between a first user device operated by a patient (e.g., user device 108) and a second user device operated by a healthcare provider (e.g., user device 112). Communication network 120 may enable one or more other devices associated with the first user device (e.g., such as nurses, aides, social workers, parents, adult children, etc.) and/or the second user device (e.g., nurses, assistants, other doctors, administrators, etc.) to connect to the communication session.

Communication network 120 may include one or more processing devices (e.g., computing devices, mobile devices, servers, databases, etc.) configured to operate together to provide the services of communication network 120. The one or more processing devices may operate with a same local network (e.g., such as a local area network, wide area network, mesh network, etc.) or may be distributed processing devices (e.g., such a cloud network, distributed processing network, or the like). User device 108 and user device 112 may connect to communication network 120 directly or through one or more intermediary networks 116 (e.g., such as the Internet, virtual private networks, etc.).

The first user device or the second user device may request a new communication session using communication session manager 124. The request may include an identification of one or more user devices are authorized to connect to the communication session. The request may also include other parameters such as user profile data (associated with the first user device or the second user device, such as, but not limited to an identification of a user of the user profile, a user identifier, user devices operated by the user), a purpose for establishing the communication session, a start time of the communication session, an expected duration of the communication session, settings of the communication session (e.g., audio channel settings, video channel settings, collaborative window settings, wrapper settings, etc.), combinations thereof, or the like.

Communication session manager 124 may then instantiate a new communication session for the first user device and/or the second user device. The new communication session may include one or more environments for the user devices connected to the communication session. The environment may include user interfaces, wrappers, resources, application programming interfaces, etc. configured to extend the functionality of the communication session. The communication session manager 124 using ML core process 132 may provision one or more machine-learning models to enable any of the extended functionality. The one or more machine-learning models may be configured to provide natural language processing (e.g., such as a large language model, bi-directional transformers, zero/few shot learners, deep neural networks, etc.), content generation (e.g., using large language models, deep neural networks, generative adversarial networks, etc.), single variate or multivariate classifiers (e.g., k-nearest neighbors, random forest, logarithmic regression, decision trees, support vector machines, gradient decent, etc.), image processing (e.g., using deep neural networks, convolutional neural networks, etc.), sequenced data processors (e.g., such as recurrent neural networks, etc. capable of processing datasets organized according to a taxonomic sequence), and/or the like. The one or more machine-learning models may be configured to process natural language communications (e.g., such as gestures, verbal, textual, etc.) to provide real-time translations and/or transcriptions, generate natural language communication capable of autonomous communication (e.g., a communication bot) or content generation (e.g., such generating natural language responses to requests for information, etc.), user authentication (e.g., to ensure users connected to the communication session are authorized to do so), and/or the like.

Communication session manager 124 may authenticate each user device that connects to the new communication session using user authentication 128. User authentication 128 may ensure that the user of a connected user device corresponds to an authorized user. To avoid exposing personal identifiable information or medical information, user authentication 128 may compare abstracted features associated with the user to corresponding abstracted features associated with an authorized user. In some instances, the abstracted features may include an abstracted representation of a username, password, token, public/private key, and/or the like. Communication session manager 124 may distribute passwords, tokens, public/private keys, and/or the like with an invitation to connect to the new communication session. In some instances, the abstracted features may include biometric features of a user of a user device to be authenticated such as physical features, vocal features, and/or the like. For example, using monocular depth estimation facial features can be extracted based on a relative distance of a representation of the facial feature (e.g., in a video frame) from the camera that captured the representation. The relative distances may be used to determine if the user a user device corresponds to a known, authenticated user by comparing the relative distances to stored relative distances.

User authentication 128 may obtain one or more video frames and/or one or more audio segments received from a user device to be authenticated. The one or more video frames may be generated using a camera of the user device and include a representation of a user of the user device. The audio segments may include a representation of a voice of the user. User authentication 128 may transmit a request to machine-learning (ML) core process 132 to process the video and/or audio. For example, using a first machine-learning model of machine-learning models 148, a depth map may be generated using a video frame including a representation of a user. The depth map may include a distance value for each pixel of the video frame corresponding to a predicted distance of a point in the environment represented by the pixel from the camera that captured the video frame. User authentication 128 may use the depth map to distinguish pixels corresponding to the user from pixels corresponding to the background (e.g., based on the pixels representing the user being generally closer than pixels representing the background). User authentication 128 may then determine relative differences in distances between one or more pixels to determine a relative depth of one or more facial features. The relive differences in distances may be abstracted features that may be used to determine whether the user represented by the video data is authenticated by comparing the abstracted features to abstracted features of an authenticated user.

Using a second machine-learning model, user authentication 128 may process audio segments including a representation of the user's voice. The second machine-learning model may process the audio segments to derive abstracted features associated with the audio segments, the second machine-learning model may be configured to identify pitch, tone, speech velocity, pause frequency and length, diction, accent, language, etc.) of the audio segment represented as a sequence of numerical values. The abstracted features can be compared to historical abstracted features of an authenticated user to determine if the user associated with the abstracted features is the same user as the user associated with the historical abstracted features.

Communication session manager 124 may pass communications extracted over the communication session to ML core process 132 process the communications using the one or more machine-learning models. ML core process 132 may monitor one or more machine-learning models configured to provide the services of the communication network. ML core process 132 may train new machine-learning models, retrain (or reinforce) existing machine-learning models, delete machine-learning models, and/or the like. Since ML core process 132 manages the operations of a variety of machine-learning models, each request to ML core process 132 may include an identification of a particular machine-learning model, a requested output, or the like to enable ML core process 132 to route the request to an appropriate machine-learning model or instantiate and train a new machine-learning model. Alternately, ML core process 132 may analyze data to be processed that is included in the request to select an appropriate machine-learning model configured to process data of that type.

If ML core process 132 cannot identify a trained machine-learning model configured to process the request, then ML core process 132 may instantiate and train one or more machine-learning models configured to process the request. Machine-learning models may be trained to process a particular input and/or generate a particular output. ML core process 132 may instantiate and train machine-learning models based on the particular data to be processed and/or the particular output requested. For example, user sentiment analysis (e.g., user intent, etc.) may be determined using a natural language processor and/or a classifier while image processing may be performed using a convolutional neural network.

ML core process 132 may select one or more machine-learning models based on characteristics of the data to be processed and/or the output expected. ML core process 132 may then use feature extractor 136 to generate training datasets for the new machine-learning models (e.g., other than those models configured to perform feature extraction such as some deep learning networks, etc.). Feature extractor 136 may define training dataset using historical session data 140. Historical session data 140 may store features from previous communication sessions. In some instances, the previous communication sessions may not involve the user of the first user device or the user of the second user device. Previous communication sessions may include manually and/or procedurally generated data generated for use in training machine-learning models. Historical session data 140 may not store any information associated with healthcare providers or patients. Alternatively, historical session data 140 may store features extracted from communication session involving the user of the first user device, the user of the second user device, and/or other patients and/or other healthcare providers.

Feature extractor 136 may extract features based on the type of model to be trained and the type of training to be performed (e.g., supervised, unsupervised, etc.) from historical session data 140. Feature extractor 136 may include a search function (e.g., such as procedural search, Boolean search, natural language search, large language model assisted search, or the like) to enable ML core process 132, an administrator, or the like to search for particular datasets within historical session data 140 to improve the data selection for the training datasets. Feature extractor 136 may aggregate the extracted features into one or more training datasets usable to train a respective machine-learning model of the one or more machine-learning models. The training datasets may include training datasets for training the machine-learning models, training datasets to validate an in-training or trained machine-learning model, training datasets to test a trained machine-learning model, and/or the like. The one or more training datasets may be passed to ML core process 132, which may manage the training process.

Feature extractor 136 may pass the one or more training datasets to ML core process 132 and ML core process 132 may initiate a training phase for the one or more machine-learning models. The one or more machine-learning models may be trained using supervised learning, unsupervised learning, self-supervised learning, or the like. The one or more machine-learning models may be trained for a predetermined time interval, a predetermined quantity of iterations, until one or more target accuracy metrics have exceeded a corresponding threshold function (e.g., accuracy, precision, area under the curve, logarithmic loss, F1 score, weighted human disagreement rate, cross entropy, mean absolute error, mean square error, etc.), user input, combinations thereof, or the like. Once trained, ML core process 132 may validate and/or test the trained machine-learning models using additional training datasets. The machine-learning models may also be trained at runtime using reinforcement learning.

Once the machine-learning models are trained, ML core process 132 may manage the operation of the one or more machine-learning models (stored with other machine-learning models in machine-learning models 148) during runtime. ML core process 132 may direct feature extractor 136 to define feature vectors from received data (e.g., such as the video data, audio segments from the first user device or the second user device, content of a collaborative window such as collaborative window 204, etc.). In some instances, ML core process 132 may facilitate generation of a feature vector each time there is a change in the communication channel (e.g., a change in video from a user device, an audio segment is transmitted over the communication channel, content is added or removed from a user interface of the communication session (e.g., such as patient interface 200, provider interface 232, temporary environment 252, or any other interface displayed to a user device), content is modified from a user interface of the communication session (e.g., such manipulating an image, etc.), a timestamp relative to a start time of the communication session, or the like. ML core process 132 may continually execute the one or more machine-learning models to generate corresponding outputs. ML core process 132 may evaluate the outputs to determine whether to manipulate a user interface of the communication session based on the output (e.g., post automatically generated content, modify a word wall weights or words, add/remove suggested questions, initiate an automated conversation with a bot, provide information associated with keywords spoken during the communication session, etc.).

For example, ML core process 132 may detect a new audio segment communicate over the communication session. ML core process 132 execute a machine-learning model (e.g., such as a recurrent neural network) to process the audio segment to determine the words within the audio segment (if any) and a sentiment (e.g., a predicted meaning of the individual words or the words as a whole). ML core process 132 may execute another machine-learning model (e.g., such as a classifier, a large language model and/or transformer, a generative adversarial network, etc.), to generate content corresponding to the words and/or sentiment that can be provided to a user device. For instance, the words may include “My knee is swollen and painful” with a sentiment of “symptoms.” The other machine-learning model may process the words and sentiment to generate content for patient interface 200 such as information about ailments associated with knee pain and knee swelling, home treatments that may alleviate symptoms and/or improve mobility, possible questions that can be asked to the healthcare provider, etc. ML core process 132 may also use the other machine-learning model to generate content for provider interface such as symptoms, suggested follow up questions regarding the degree of swelling or the intensity of the pain, links for additional information associated with knee pain or knee swelling, links associated with ailments associated with knee pain or knee swelling, etc.

ML core process may direct feature extractor 136 to define other feature vectors to process other data using machine-learning models of machine-learning models 148 in parallel with the aforementioned machine-learning models to provide other resources of communication network 120. ML core process 132 may execute any number of machine-learning models in parallel to provide the functionality of a communication session.

Communication session manager 124 may update the interfaces (e.g., patient interface 200, provider interface 232, temporary environment 252, and/or any other interface presented to user devices during a communication session) in real-time. Content may be received from a particular user device associated with a collaborative window (that may be viewable by all user devices connected to the communication session) via a drag and drop over the collaborative window, an upload link, or the like. Communication session manager 124 may process the received content into a format that can be embedded into the collaborative window and update the collaborative window (or the entire interface) enabling other user devices connected to the communication session to view the content in a same manner as provided by the particular user device. Communication session manager 124 may also receive outputs from machine-learning models 148 via ML core process 132 and determine whether to update the interfaces using the output and how to update the interfaces using the output (e.g., where to present generated content within the interface, what fonts and font sizes to present generated content, whether generated content is to expire after a time interval and be removed from the interface, whether the content is related to and should presented proximate to other content, etc.). Communication session manager 124 may continuously update and the interfaces to present a dynamic, collaborative interface to each user device through the communication session. When the communication session terminates, communication session manager 124 may store the interfaces with an identifier associated with the communication session for further processing and/or replay by a user device of the communication session.

FIG. 2A illustrates an example user interface of the communication network presented to user devices of a communication session according to aspects of the present disclosure.

Patient interface 200 may include one or more elements accessible by a first user device over the duration of a communication session. The first user device may be operated by a patient or an entity associated with the patient (e.g., nurse, assistant, aide, medical proxy, etc.). The one or more elements may contain content and/or links to content pertaining to the communication session. The one or more elements may be selected by a healthcare provider, a machine learning algorithm, one or more other user devices, associated health system settings, any combination thereof, or the like. The one or more elements within patient interface 200 may be specific to a particular communication session. The one or more elements may be visual or may contain interactive portions, wherein the user device may “click” or “select” specific portions to view additional content or where the user device may provide information to one or more other devices connected to the communication session.

For example, collaborative window 204 may contain one or more objects. The objects may be selected automatically (e.g., by a machine-learning model, rules engine, etc.), by the second user device, and/or the first user device. For example, the second user device may present objects to the first user device pertaining to an ailment (e.g., symptoms, diagnosis, treatments, historical facts, lifestyle or dietary interactions, etc.). In another illustrative example, objects pertaining to test results and/or anatomic scans may be presented within collaborative window 204. The first user device and/or the second user device may review the shared content simultaneously in real-time. In some examples, the first user device may also generate presentations containing visual content and present them on collaborative window 204 (e.g., uploading images or video segments, drawing using a curser, transmitting alphanumeric text, etc.).

Toolbar 228 may contain tools for a user device to manipulate collaborative window 204. The tools may include, but are not limited to, a pencil tool, a shape tool, a text tool, a “mute” button, a “camera” button, and an “end call” button. The tools described herein may allow the user device to generate additional content for display to one or more user devices connected to the communication session. For example, the user device may use a “pencil” tool to circle a particular element of a set of content.

In some examples, camera view 224 may display a real-time representation a user (e.g., such as a face or upper body of the patient or another user associated with a user device connected to the communication session) within collaborative window 204 if a user device is equipped with a camera. Camera view 224 may present a representation (e.g., a sequence of video frames captured by a camera of the first user device) of a user operating the first user device. If the first user device is equipped with multiple cameras (e.g., a camera pointed away from the user and a camera pointed towards the user), then camera view 224 may display an output from one of the cameras according to settings of the user device. Camera view 224 may vary in size, depending on the size of the screen of the user device connected to the communication session, the content currently displayed in collaborative window 204, communication session parameters associated with the communication session, any combination thereof, or the like. Camera view 224 may change in size dynamically or may be changed by a user device connected to the communication session. Camera view 224 may be eliminated entirely from collaborative window 204, either temporarily or for the entirety of the duration of the respective communication session to enable access to an additional area of collaborative window 204, prevent a distraction, etc. Removing camera view 224 may include reducing a size of camera view 224 to a zero-by-zero-pixel window or by causing the camera view 224 to become transparent. By maintaining the window in a reduced size and/or increased transparency may reduce the latency when bringing camera view 224 (e.g., resizing camera view 224 or reducing the transparency of camera view 224).

Camera view 224 may display a particular user associated with a user device connected to the communication session according to the user device currently outputting audio. For example, if three user devices are connected to a communication session, and the three user devices are associated with three respective individuals—a healthcare provider, a patient, and the patient's guardian—camera view 224 may display the healthcare provider while the healthcare provider is providing a diagnosis, but may switch to a display of the patient's guardian if the patient's guardian begins to ask a question. In some examples, the communication network may selectively determine whether video transmitting from a user device should be displayed by camera view 224. For example, the communication session may determine not to display a video feed associated with the first user device, even if that first user device is outputting audio. For example, using the same communication session as above, camera view 224 of the second user device will not show the video feed of the healthcare provider, even if the healthcare provider is the one speaking. However, camera view 224 of the user devices associated with the patient and the patient's guardian may display the healthcare provider.

User interface wrapper 220 may comprise a graphic overlay that frames collaborative window 204 and displays content. User interface wrapper 220 may contain branding messaging, including, but not limited to, the associated health system logo, the communication network logo, the associated health clinic logo (e.g., a health clinic where an associated healthcare provider may actively practice), other content (e.g., advertisements, services provided by the healthcare provider or health clinic, etc.), and/or communication session appointment information (e.g., date, time, etc.). The content displayed on user interface wrapper 220 may cycle throughout the communication session. For example, the other content displayed on user interface wrapper 220 may change every ten seconds in a consistent loop, wherein the same five sets of content are shown in the same order repeatedly. In some instances, the other content may be selected based on the communication session (e.g., such as, but not limited to, based on words said by the healthcare provider, words said by the patient, information presented within collaborative window 204, combinations thereof, or the like).

In some examples, the user interface wrapper 220 may be extended to contain additional content, including healthcare provider information 216, key phrase panel 212, and question prompt 208. Healthcare provider information 216 may contain information pertaining to the current communication session. In some examples, it may include the name of the healthcare provider connected to the communication session and/or the medical practice area of the healthcare provider. In some examples, healthcare provider information 216 may include a location in which the healthcare provider is located (e.g., “Boston, Massachusetts”, an address of an office of the healthcare provider, etc.). Healthcare provider information 216 may also include an upload link. A user device and/or another user device connected to the communication session may upload documents, photos, documentation, surveys, etc. requested by the healthcare provider using the upload link. The user device may select whether information uploaded via the upload link is to be displayed within collaborative window 204 or sent directly to a particular user device connected to the communication session (e.g., the healthcare provider, etc.). The healthcare provider may request this information during or prior to the communication session. At time of upload, the communication network transmits the data in real time to a location accessible by the second user device, such as a network folder, cloud drive, shared storage drive, any combination thereof, or the like. The location accessible by the second user device may contain the entirety of the documentation provided by the user device, including, but not limited to, uploads from prior communication sessions.

Key phrase panel 212 may be generated by communication network 120 with a machine-learning algorithm and/or a natural language processing (“NLP”) machine-learning model. The NLP machine-learning model may receive transmitted audio data from the user devices connected to the communication session and identify one or more words and/or phrases appearing in the transmitted audio data. The NLP machine-learning model may transform the words and/or phrases into a graphic display, as shown in FIG. 2A, with the larger word/phrase size indicating more frequent appearances in the transmitted audio data. In some examples, the first user device may select and/or click the words and/or phrases comprising key phrase panel 212 to be redirected to a second user interface displaying additional information pertaining to the word/phrase. The second user interface may include a website, an internal document from the associated health system and/or the healthcare provider, a presentation, an application or service, any combination thereof, or the like. The other page may be generated by a machine learning algorithm trained to select relevant materials for patients (e.g., such as a large language model, generative adversarial network, or the like). In some examples, the communication network may track the activity and/or interactions between the first user device and patient interface 200. The second user device may receive a notification including data pertaining to the activity and/or interactions of the first user device. For example, this data may be analyzed to indicate which topics the healthcare provider should revisit for the patient before the conclusion of the communication session or during a subsequent communication session. Key phrase panel 212 may be an abstract design, as shown in FIG. 2A, a list, or another visual representation of topics covered over the duration of the communication session.

In some instances, user interface wrapper 220 may contain question proposal 208. Question proposal 208 may utilize a similar NLP machine-learning system as key phrase panel 212. The NLP system may receive transmitted audio data from the user devices connected to the communication session and identify one or more words appearing in the transmitted audio data. A machine-learning algorithm may generate a set of questions directed towards the one or more words. The set of questions may be presented on the user device and the patient may select questions to ask the healthcare provider. The machine-learning algorithm may reference historical communication sessions between the one or more user devices connected to the communication session and/or all historical communication sessions conducted via communication network 120.

At the conclusion of the communication session, patient interface 200 may automatically close and the communication network may automatically redirect the user device to another location within the communication network or other connected webpage. Collaborative window 204, key phrase panel 212, question proposal 208, and/or other content displayed on user interface wrapper 220 may be made accessible to the one or more user devices connected to the communication session after the conclusion of the communication session via a transmitted link, a patient portal, the telehealth environment, an application or service, a webpage, any combination thereof, or the like.

FIG. 2B illustrates an example user interface of the communication network presented to user devices of a communication session according to aspects of the present disclosure.

Provider interface 232 may contain one or more elements accessible by a second user device over the duration of a communication session. The second user device may be operated by a healthcare provider. The one or more elements may contain content and/or links to content pertaining to the communication session. Provider interface 232 may differ from a user interface associated with a different user device (e.g., patient interface 200 described in FIG. 2A). The one or more elements may be selected by the healthcare provider, a machine learning algorithm, one or more other user devices, associated health system settings, any combination thereof, or the like. The one or more elements within provider interface 232 may be specific to the communication session. The one or more elements may be visual or may contain interactive elements, wherein the user device may “click” or “select” specific elements to view additional content or where the second user device may provide information one or more other devices connected to the communication session. Provider interface 232 may include elements previously described in FIG. 2A (e.g., patient interface 200) including, but not limited to, collaborative window 204, toolbar 228, camera view 224, and user interface wrapper 220. The aforementioned elements may include any of the features as previously described as well as the modifications and/or additions described herein.

In some examples, the user interface wrapper 220 may include content that may be relevant to the healthcare provider such as, but not limited to, patient information 236, patient history (not shown), content library 240, preliminary assessment 244, and question recommendations 248. User interface wrapper 220 associated with the second user device (e.g., the user device operated by the healthcare provider) may not include certain elements, including, but not limited to, advertisements, a word cloud (e.g., key phrase panel 212 described in FIG. 2A), and/or healthcare provider information (e.g., healthcare provider information 216 described in FIG. 2A).

Patient information 236 may display a high-level summary of the patient for reference by the healthcare provider. The data displayed in patient information 236 may be received from the medical history of the patient, intake paperwork, user input (e.g., from the patient, healthcare provider, etc.), any combination thereof, or the like. Patient information 236 may include the patient's name, gender/sex, age, weight, relevant ailment, information that may assist the healthcare provider over the duration of the communication session, any combination thereof, or the like. The data presented in patient information 236 may be selected by the healthcare provider, an individual directed by the healthcare provider, the associated health system, a machine learning algorithm, any combination thereof, or the like. The data presented in patient information 236 may be unique to the communication session. During a subsequent communication session, patient information 236 may include different information based on the patient and the subsequent communication session. Patient information 236 may also comprises a link for the user device to select and/or view documents related to the patient, such as a medical history, submitted photos, video (if the first user device is transmitting video), contact information, insurance information, any combination thereof, or the like. In some examples, the communication network may provision a first machine-learning model capable of conducting facial analysis to supplement information provided in patient information 236. The video stream of the patient shown via camera view 224 on the second user device may be received by the first machine-learning model. The first machine-learning model may conduct facial analysis of the patient and infer one or more characteristics about the patient, including pain, confusion, interest, contentment, anger, frustration, sadness, etc.

Content library 240 may be a storage location accessible by the second user device via the telehealth environment, communication session environment, provider interface 232, any combination thereof, or the like. Content library 240 may contain content selected by the healthcare provider that can be selected, accessed, or presented by the second user device. In some instances, content library 240 may be access restricted to prevent access to data stored in content library 240 by users other than the healthcare provider. The storage location of content library 240 may be a local storage device, cloud storage, shared network drive, any combination thereof, or the like. Content library 240 may be store content associated with a particular communication session or associated with a particular patient. For example, content library 240 be store different content during different communication session involving a same patient and healthcare provider.

The second user device may select content (e.g., data, information, files, etc.) to be stored in content library 240 and display selected content from content library 240 through collaborative window 204. Data can be stored in the storage location before the communication session, during the communication session, and/or after the communication session. The second user device may select data for storage in a local storage medium (e.g., local storage device, cloud storage, shared network drive, any combination thereof, or the like), data stored in a remote storage medium or network (e.g., cloud network, webpage, storage area network, database, server), data from collaborative window 204 (e.g., data provide by the patient or by the healthcare provider), or the like for storage in content library 240. For example, content library 240 may include presentations, informative videos, diagrams, charts, tables, photos, scans, test results, and/or any content that may be relevant to the communication session. In some instances, content library 240 may include data generated by a machine-learning algorithm (e.g., such as a large language model, classifier, etc.) trained to identify relevant content for the communication session according to a number of factors, including, but not limited to, patient data, medical history, communication session history, healthcare provider practice area, the communication session, any combination thereof, or the like. In some examples, the machine-learning algorithm may dynamically generate recommendations withing content library 240 that pertain to the communication session at a given point. In some examples, content library 240 may be searched with one or more natural language commands from the healthcare provider. A machine-learning model trained to interpret natural language may receive the one or more natural language commands and output relevant data. For example, the healthcare provider may request, “show me the results of the patient's bloodwork,” and a second machine-learning model may receive the request, query the storage location, and output the most recent bloodwork results associated with the patient.

Preliminary assessment 244 may contain relevant data associated with the patient. Preliminary assessment 244 may display one or more physical characteristics of the patient (e.g., height, weight, etc.), lifestyle habits of the patient (e.g., exercise, smoking, drinking, etc.), and/or information related to the reason for the communication session (e.g., injury details, symptoms, etc.). The data may be populated using information obtained during the patient intake process, from forms shared from another healthcare provider (e.g., with consent of the patient), medical history of the patient, generated using a machine-learning algorithm trained to infer additional details about a patient, any combination thereof, or the like. Preliminary assessment 244 may be dynamic and may change throughout the duration of the communication session according to the conversation between the healthcare provider and the patient. Preliminary assessment 244 may add additional data and/or may change existing data (e.g., change weight from 160 lbs. to 165 lbs.).

Question recommendations 248 may generate possible prompts to the second user device. The second user device may present a possible prompt to the first user device (e.g., user device operated by the patient) and/or other user device connected to the communication session. The prompts may be generated by a third machine-learning algorithm trained to aggregate patient data and historical data to articulate pertinent questions. In some examples, the possible prompts may be populated by the healthcare provider, or someone directed by the healthcare provider. The possible prompts may also be generated by any combination of the above. Question recommendations 248 may dynamically change according to the audio transmitted over the duration of the communication session. An NLP system may identify if a possible prompt has been recognized within the transmitted audio (e.g., communicated by a patient or healthcare provider) and may remove it from question recommendations 248. In some instances, the third machine-learning algorithm may prioritize the possible prompts (e.g., putting the most important prompt at the top of the list) and may dynamically alter the prioritization of the possible prompts according to the transmitted audio of the communication session examined by the NLP system. For example, the third machine-learning algorithm may generate the prompt, “do you remember a distinct date or event when the knee pain started?” As the communication session progresses, the machine-learning algorithm may remove the prompt from the list within question recommendations 248 if the healthcare provider asks the question (or a similar question) or if the patient provides an answer to the question, or the prompt may be moved downward on the list as the conversation between the healthcare provider and the patient gradually moves past introductory conversation and general background information. Alternatively, if the third machine-learning algorithm may move the prompt up the list to ensure the healthcare provider addresses the prompt.

At the conclusion of the communication session, provider interface 232 may automatically close and the communication network may automatically redirect the user device to another location within the communication network or other connected webpage. Content library 240, preliminary assessment 244, question recommendations 248, and/or other content displayed on user interface wrapper 220 may be made accessible to one or more user devices connected to the communication session after the conclusion of the communication session via a transmitted link, a patient portal, the telehealth environment, an application or service, a webpage, any combination thereof, or the like.

FIG. 2C illustrates an example temporary environment of the communication network configured for a particular user device according to aspects of the present disclosure.

Temporary environment 252 may be an interim environment for user devices before a communication session begins or before a particular user device connects to the communication session (e.g., such as the user devices operated by the healthcare provider). For example, a first user device (e.g., user device associated with the patient) may be connected to temporary environment 252 while waiting for a second user device (e.g., user device associated with the healthcare provider) to connect to the communication session. Temporary environment 252 may allow the first user device to complete administrative tasks (e.g., filling out forms, providing information, etc.), access information associated with the communication session or healthcare provider, etc.) while waiting for the second user device to connect to the communication session. Temporary environment 252 may include a user interface comprising one or more elements, including, but not limited to, temporary environment wrapper 268, chatbot interface 264, task list 256, and/or administrative tasks 260. The one or more elements may be selected by connected user devices, the healthcare provider, a machine-learning algorithm, associated health system, any combination thereof, or the like. The one or more elements within temporary environment 252 may be unique to the communication session. The one or more elements may be visual or may contain interactive elements, wherein the first user device may “click” or “select” specific portions to view additional content or where the first user device may provide information to one or more other devices connected to the communication session. In some examples, temporary environment 252 may prompt the first user device to provide remote device data. The remote device data may include, but is not limited to, smartwatch data, smartphone data, wearable health tracker data, heart rate monitor data, etc. The first user device may permit the communication network to access the remote device data or may restrict access to one or more data points, sources, time periods, etc.

Temporary environment wrapper 268 may comprise a graphic overlay that displays content and/or the one or more elements within temporary environment 252. Temporary environment wrapper 268 may include healthcare provider information (e.g., healthcare provider information 216), the associated health system logo, the communication network logo, and/or any element and/or aspect of user interface wrapper 220 of FIG. 2A. The content display on temporary environment wrapper 268 may cycle throughout a time interval corresponding to the time over which the user device is connected to the temporary environment 252.

Task list 256 may include an identification of a set of tasks that may be completed by the user device within the time interval. The tasks of task list 256 may be related to the current communication session, historical communication sessions, identified ailments, insurance information of the patient, a tutorial of temporary environment 252 and/or the communication network, any combination thereof, or the like. Tasks of task list 256 may redirect the user device to an appropriate platform to complete the individual item (e.g., using pointers, hyperlinks, application programming interfaces, remote procedure calls, etc.). The tasks may be generated by the healthcare provider, the associated health system, and associated health clinic, a nurse or other provider, the patient, a machine-learning algorithm, any combination thereof, or the like. Communication network 120 may monitor information received from user devices to determine if information associated with task has been received completing the task. Communication network 120 may automatically remove the task from task list 256. For example, communication network 120 may determine that the user device uploaded a photo, thus a “check mark” will appear next to the appropriate task. The communication may continue before the tasks of task list 256 are completed.

Chatbot 264 may be a virtual chat platform enabled by generative artificial intelligence or another machine-learning algorithm (e.g., large language models, natural language models, etc.). The user device may input a query into chatbot 264, and chatbot 264 may output an appropriate response according to configurable restrictions that can be applied to chatbot 264 (e.g., excluding confidential information, diagnosis, and other medical opinion related information, etc.). The configurable restrictions may be set by the associated health system, the associated health clinic, the healthcare provider, hardcoded, any combination thereof, or the like. Conversations between chatbot 264 and the user device may be saved in local memory and/or may be distributed to the user device using a digital messaging service (e.g., email, text messaging, direct messaging, instant messaging, as a file in the telehealth environment, etc.). Data exchanged between chatbot 264 and the user device may be encrypted and/or secured by another manner to comply with the Health Insurance Portability and Accountability Act (HIPAA), healthcare regulations, privacy policies, and other regulations and/or policies.

Administrative tasks 260 may include an identification of potential administrative actions the user device may complete. The tasks shown in administrative tasks 260 may be related to various tasks pertaining to the patient's medical records, contact information, communication session scheduling, requesting documentation or records, any combination thereof, or the like. A task in administrative tasks 260 may be selected to redirect the user device to an appropriate platform to complete the individual item (e.g., using pointers, hyperlinks, application programming interfaces, remote procedure calls, etc.). The tasks may be generated by the healthcare provider, an associated health system, and associated health clinic, a nurse or other provider, the patient, a machine learning algorithm, any combination thereof, or the like.

If the first user device is executing operations of temporary environment 252 when and the second user device connects to the communication session, the first user device may receive a notification indicating that the temporary environment 252 will be terminated. In some instances, communication network 120 may automatically redirect the first user device to the communication session. In other instances, the first user device may be presented with a “countdown” to be automatically redirected (e.g., once the user device operated by the healthcare provider connects, the user device has 30 seconds to join the communication session or the user device will be automatically joined). In still yet other instances, communication network 120 may redirect the first user device upon completion of an operation by the first user device (e.g., selecting a “join session” button, scheduling a follow-up session, completing intake forms, etc.). Once temporary environment 252 terminates, user devices may be prevented from accessing temporary environment 252. The one or more elements presented in temporary environment 252 may be accessible in a different environment, such as patient interface 200, telehealth environment, or another interface associated with the patient within the communication network.

FIGS. 2A-2C illustrate various example user interfaces of the communication system. The example user interfaces are merely examples implementations of the contents of this specification. FIGS. 2A-2C are in no way intended to limit the scope of this specification.

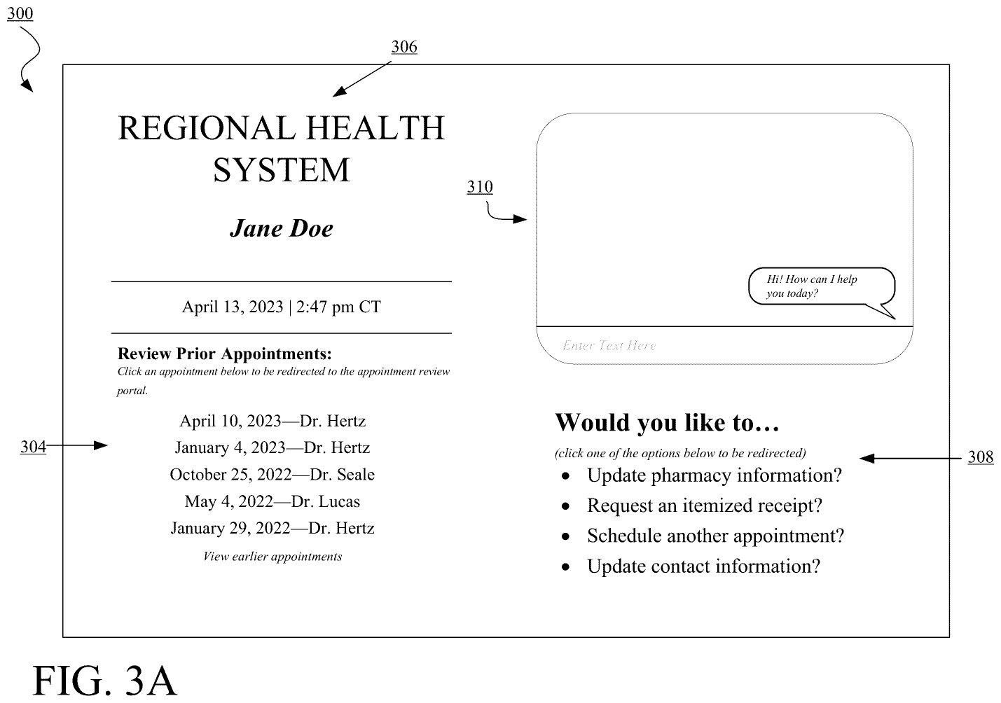

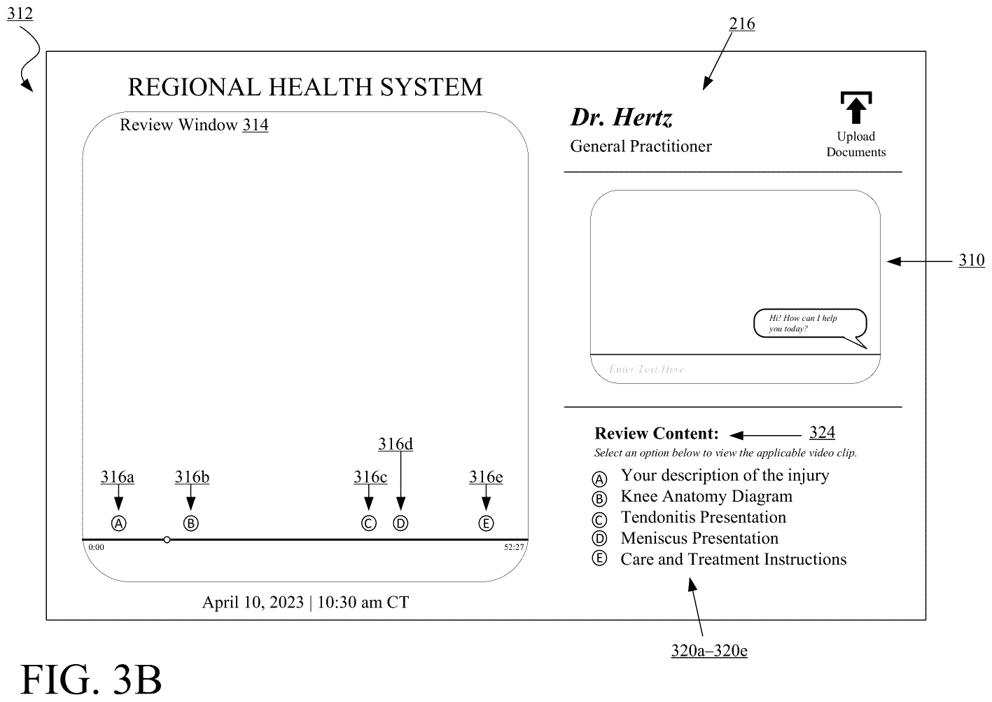

FIG. 3A illustrates an example post-session environment of a communication network configured for a particular user device according to aspects of the present disclosure. Post-session environment 300 may be a secured environment (e.g., such as a virtualized environment, container, partition, etc.) configured enable execution of components of the communication network. In some instances, post-session environment 300 may be sub-environment within the telehealth environment such that once connected to the telehealth environment, a user device may navigate to telehealth environment to connect to post-session environment 300. In other instances, post-session environment 300 may be a separate environment from the telehealth environment such that post-session environment 300 may be accessed separately from the telehealth environment.

Post-session environment 300 may be a secured environment to prevent unauthorized access to sensitive user information (e.g., personal identifiable information, health records and/or health related information, etc.). Post-session environment 300 may be instantiated as a local environment within a device or as a distributed environment within one or more devices. Post-session environment 300 may be a virtualized or other self-contained environment to prevent information from being accessible by other processes of the device or devices that facilitate post-session environment 300 (e.g., such as an operating system, other applications executing on the devices, malicious software, etc.). Post-session environment 300 may use access credentials such as a username and passcode, one or more encryption keys, tokens, any combination thereof, or the like to prevent unauthorized users from access a particular post-session environment 300.

Post-session environment 300 may be configured for a user to access previous communication sessions associated with the user, features of the communication network outside of communications sessions, related information associated with the user or the communication network, etc. Post-session environment 300 may be customized for individual users based on previous communication sessions associated with a user, one or more healthcare providers and/or healthcare systems associated with the previous communication sessions, communication network, user input, and/or the like. For instance, post-session environment 300 may include a graphical user interface configured to present information and provide access to features of the post-session environment. The graphical user interface may be customized by changing the look and feel (e.g., colors, icons, orientation, layout, size, etc.), adding and/or removing functions, adding and/or removing information, to improve the distribution of information to the user and increase interactivity with the post-session environment. post-session environment 300 may be automatically customized (e.g., using one or machine-learning models using historical communication sessions associated with the user) and/or manually (e.g., via user input from the user, a healthcare provider, a user associated with a healthcare system, etc.).

Post-session environment 300 may facilitate access to information and/or functions of the communication network to user devices outside of communication sessions. For example, post-session environment 300 instantized for a first user (e.g., such as a patient, etc.) may provide access to previous communication sessions associated with the first user device (e.g., such as a searchable representation of the audio and/or video channel of the communication sessions), access to objects presented in a collaborative window of the communications sessions, present information derived from the communication session (e.g., using various machine-learning models, etc.), access communication bots configured to automate communications and/or simulate a healthcare provider or healthcare system, provide tools to facilitate subsequent communication sessions with healthcare providers or healthcare systems, access records (e.g., such as electronic medical records (EHR), etc.), access various machine-learning-based tools (e.g., natural language processors, classifiers, etc.), and the like.

In an illustrative example, a communication network (e.g., communication network 120 of FIG. 1) may instantiate post-session environment 300 for a first user device. The first user device may be operated by a first user (e.g., a patient, a user associated with a patient, etc.). The communication network may instantiate a post-session environment 300 when the first user device registers with the communication network, the first time the first user device and/or the user thereof connects to a communication session or disconnects from a communication session, upon request by the first user device or a healthcare provider associated with the first user device, etc. For example, upon terminating a communication session, the communication network may connect the first user device to post-session environment 300 instantiated for the first user device. The communication network may store the audio and/or video channel of a communication session to enable reconstructing a representation (e.g., a recording, etc.) of the communication session in a particular format. The format may use a particular compression algorithm and/or container to secure the reconstructed representation from being accessible outside the communication network.

Post-session environment 300 may enable the first user device to replay any portion of the communication session such as the audio channel and/or the video channel of the communication session. Post-session environment 300 may include one or more controls to enable presentation of particular portions of the communication session (e.g., such a portion of the communication session in which a particular diagnosis or treatment was discussed, etc.), replay the communication session in real time or in accelerated time, access objects of the collaborative window associated with particular contexts (e.g., such as particular diagnosis, treatments, etc.), and/or the like.

Post-session environment 300 may include review wrapper 306, appointment history 304, actions 308, and chat interface 310. Review wrapper 306 may be container through which one or more graphical user interfaces or graphical user interface elements can be displayed. Review wrapper 306 may present content associated with a healthcare provider and/or healthcare system such as, but not limited to, a logo associated with the healthcare provider or healthcare system, a logo associated with the communication network, a logo associated with a health clinic logo (e.g., a health clinic where an associated healthcare provider may practice), other content (e.g., a name of the user or the first user device, a current date and/or time, etc.), communication session information, etc. In some examples, review wrapper 306 may contain messages from a second user device (e.g., a user device operated by a healthcare provider), messages from one or more other devices (e.g., such as device associated with a healthcare system, healthcare clinic, or the like), action items (e.g., actions to be executed by the first user device such as completed documents and/or signatures, upload insurance information, etc.), appointment reminders, any combination thereof, or the like. Review wrapper 306 may include any element and/or aspect of user interface wrapper 220 and temporary environment wrapper 268, described in FIG. 2A-2C.

Appointment history 304 may contain a list identifying previous communication sessions (e.g., such as the most recent previous communication sessions, previous communication sessions that were scheduled year to date, previous communication sessions over scheduled over the last 12 months, etc.). Each entry of the list may include one or more links that enable the first user device to access the corresponding prior communication sessions. The links may redirect the first user device to a pop-up window, an embedded media player, to a memory address storing files corresponding to the previous communication session for download, another location within the telehealth environment, a website, any combination thereof, or the like. In some examples, the first user device may use a search field to search a repository of previous communication sessions.

Actions 308 may include a list of accessible to the first user device. The communication network may define the actions included in the list based on the previous communication sessions associated with the first user device (e.g., using one or more machine-learning models, user input, and/or the like). The communication session may weigh the communication sessions based on the recency of the communication session with more recent communication sessions being weighted higher than previous communication sessions. For example, a machine-learning model (e.g., a natural language classifier, etc.). may classify words spoken during the most recent communication session as being associated with physical therapy. The communication network may include an action in the list of actions to schedule an appointment with an affiliated physical therapist. Some actions included in the list may be added based on other factors such as time, healthcare provider or healthcare system, documents uploaded to a healthcare provider or healthcare system, user input, and/or the like or may be included as a default action if no other relevant actions are to be included. For example, the list may include an “Update pharmacy information” action as a default action.

Chat interface 310 may enable text-based communications with a healthcare provider, healthcare system, healthcare clinic, other patients, combinations thereof, or the like. Chat interface 310 may include a menu from which the first user device may select one or more users (e.g., healthcare provider, other patient, etc.) or domains (e.g., healthcare system, healthcare clinic, other healthcare organization, etc.). In some instances, communications with a healthcare provider or domain may be direct communications (e.g., with the healthcare provider, a user of the domain, etc.). In other instances, communications with a healthcare provider or domain may be facilitated with a chat bot (e.g., software configured to simulate a user associated with a healthcare provider, a user of the domain, etc.). The chatbot may include one or more machine-learning models (e.g., transformers, large language models, etc.) configured to process and generate natural language communications. The first user device may select whether to communicate with a user or a chatbot. Alternatively, the healthcare provider and/or domain may determine whether a chat interface is to direct communications to a chatbot or a user. In some examples, a chat interface may default to a chatbot, but monitor communications to determine if and/or when to switch to a user. For example, the communication network may determine that communications transmitted by the chatbot are not providing sufficient answers to queries and cause the first user device to be connected to a healthcare provider. The chatbot may continue to monitor communications between the first user device and the healthcare provider to improve subsequent communications generated by the chatbot (e.g., via reinforcement learning or the like).

FIG. 3B illustrates an example post-session environment of the communication network configured for a particular user device according to aspects of the present disclosure.

The communication network may configure media player 314 to enable presentation of a previous communication session the first user device. The media player may be embedded within a graphical user interface 312 of post-session environment 300. The first user device may select a particular communication session from a list of previous communication sessions to replay (as previously described). The post-session environment 300 may generate graphical user interface 312 with the embedded media player to present the selected communication session. Alternatively, the media player may operate separately from graphical user interface 312 such as in a separate window, a webpage, application, etc.

Post-session environment 300 may receive a recording (e.g., as one or more files, a media stream, a pointer to a memory address, etc.) of the communication session after the communication session terminates (e.g., upon scheduled termination or when the last user device connected to the communication session disconnects from the communication session). The recording may include the video channel and/or the audio channel that was presented to the user device associated with post-session environment 300. The recording may also include the collaborative window and any objects presented therein such as the charts, test results, diagrams, graphics, videos, notes, etc. For example, post-session environment 300 associated with the patient may receive the audio and/or video channel of the communication session that was presented to the first user device during the communication session. Post-session environment 300 may modify the recording to customize the presentation of the previous communication session.

In some examples, post-session environment may incorporate a cryptographic digital watermark into the recordings (e.g., spatial watermarking, frequency watermarking, etc.) to prevent impermissible distribution of the recordings. The watermark may be generated using characteristics of the communication session, including, but not limited to, an identification of the communication session, a start time and/or end time of the communication session, an identification of the user devices and/or the users connected to the communication session, a location of the user devices connected to the communication session, characteristics of the patient (e.g., derived from demographic information, electronic medical records, previous communication sessions, a user profile, etc.), characteristics of the healthcare provider (e.g., derived from demographic information, medical practice and/or specialty, an medical school, a healthcare system, an employee identifier, previous communication sessions, a user profile, etc.), characteristics of the first user device and/or the second user device (e.g., IP address, MAC address, Internet service provider, hardware and/or software installed therein, etc.), characteristics of the communication session (e.g., communications provided by the patient and/or healthcare provider including keywords, diagnosis, treatments, symptoms, etc.), an identification of object presented within the communication session, combinations thereof, or the like. The post-session environment may select one or more characteristics (from the aforementioned non-exhaustive list) to use for a cryptographic function (e.g., such as a hash function or the like) that may output an encrypted version of the input characteristics. The post-session environment may then modify the recording to embed the encrypted version of the input characteristics as a cryptographic digital watermark within the recording. The watermark may be generated by modifying one or more pixels of the recording (e.g., such as a luminance or chrominance) or one or more audio segments (e.g., by modifying a frequency, pitch, phase, etc. of an audio segment).

For example, one or more pixels may be modified by increasing a luminance of the pixel to represent a first value (e.g., a 1) and decreasing the luminance of the pixel to represent a second value of the watermark (e.g., a 0). The modulated pixels may be used to define a binary code that correspond to the cryptographic digital watermark.

The cryptographic digital watermark may be used to ensure that the recording being viewed has not been altered. For example, if the recording includes information about the user that does not match the characteristics embedded into the watermark, then the post-session environment may flag the recording as corrupt or tampered with. The post-session environment may then report the video for further evaluation and possible reporting.

For example, post-session environment 300 may annotate the recording based on the content of the recording to add flags associated with particular contextual content. The communication network may provision one or more machine-learning models to generate timestamp flags 316 (e.g., a location within the recording where context content is referenced) and timestamp titles 320 (e.g., a description of timestamp flag). The one or more machine-learning models may be configured to identify key points within the recorded communication session with, for example, natural language processing (e.g., processing the audio channel and/or any exchanged communications), image processing (e.g., processing the video channel and objects of the collaborative window, etc.), and/or the like. The one or more machine-learning models may be configured to process audio and/or video segments of the communication session and generate one or more keywords referenced by the audio and/or video segments. For example, the one or more machine-learning models may classify words of the healthcare provider as “treatment plan” and may generate a “treatment” keyword. As another example, the one or more machine-learning models may classify the display of the patient's MRI scan and may generate a keyword of “MRI test results.”

Post-session environment 300 may generate one or more timestamp flags (e.g., such as timestamp flags 316a-316c) corresponding to the beginning of an audio and/or video segment associated with a particular keyword or group of keywords. The notable content may correspond to a disease, ailment, condition, diagnosis, treatment, referral, objects of the collaborative window (e.g., any object or objects associated with particular content), user selected content (e.g., by the patient, the healthcare provider, an aide, a nurse, a guardian, etc.), combinations thereof, or the like.

Timestamp flags 316 may be automatically overlayed on a progress bar of media player 314. For example, timestamp flags 316 may indicate a location within the recording where the healthcare provider was discussing important information, such as medication, treatment plans, diagnosis, etc. In some examples, post-session environment may generate titles 320 for each timestamp flag generated. Timestamp titles 320 may provide a brief description of the notable content at the corresponding timestamp flag 316. For example, timestamp title 320c corresponds to timestamp flag 316c and indicates that timestamp flag 316 marks the beginning of a presentation on tendonitis. Timestamp titles 320 may be contained within content review 324 of graphical user interface 312.

Content review 324 may contain a list of timestamp titles, presentations, documents, videos, content and/or objects shared during the original communication session, any combination thereof, or the like. Content review 324, timestamp title 320, and timestamp flags 316 may be populated and/or selected by an associated healthcare provider, clinician, doctor, patient, one or more machine-learning models, any combination thereof, or the like.

In some examples, the first user device may add custom timestamp flags and timestamp titles to media player 314 and/or content review 324. For example, the first user device may add a timestamp flag at a particular location within the record to mark a discussion of particular specialist recommended by the healthcare provider. The corresponding timestamp title may be automatically generated based on the content of the recording at the selected location. The timestamp title may be presented to the user for confirmation. Alternatively, or additionally, the first user device may provide the timestamp title.

In some examples, the first user device may transmit communications to the second user device or other device associated with the communication session and/or the communication network via chat interface 310 or one or more other communication interfaces (not shown). In some examples, the communications may contain text, images, video frames extracted from the recording, video segments extracted from the recording, audio segments extracted from the recording, timestamp information (e.g., timestamp flag 316, a custom timestamp, etc.), an identification of subject matter references in the recording, any combination thereof, or the like. In some examples, the message may be transmitted with an attachment and/or link (e.g., hyperlink, pointer, etc.) usable to access the EHR associated with the first user device, the communication session, and/or other pertinent information. In some examples, chat interface 310 may direct the communications to a machine-learning model capable of processing natural language and providing answers to healthcare questions (e.g., a large language model, etc.). For example, if the second user device is unavailable, chat interface 310 may direct a communication from the first user device to the machine-learning model. The machine-learning model may receive the communication, generate an answer, and provide the answer to the first user device.

Post-session environment 300 may export the recording and/or the modified recording to local memory of the first user device. The first user device may designate the recording, the modified recording, and/or any portion thereof as approved for sharing with one or more selected user devices (e.g., such as user devices associated with a nurse, aide, guardian, other healthcare provider, etc.), and/or the like. For example, the first user device may share the modified recording with a user device of a specialist.

In some examples, the first user device may be prompted to download content (e.g., transcripts, diagrams, graphs, charts, pictures, websites, the recording, the modified recording, etc.) associated with the communication session being reviewed. For example, the first user device may be prompted to download a chart added to the collaborative window during the communication session by the second user device. For example, a diagram of a knee may be added to the collaborative window during the communication session. While the first user device is replaying the recording of the communication session at the time where the diagram is displayed, post-session environment 300 may query the first user device as to whether the diagram should be downloaded to the first user device. If the prompt is selected affirmatively, the diagram may be downloaded into local memory of the first user device, cloud storage, etc.

In some examples, post-session environment 300 may derive additional information from the recording. For example, post-session environment 300 may generate a visual transcript of communications transmitted over the audio channel of the communication session. Post-session environment 300 may execute a machine-learning model to process audio segments or video segments (e.g., American Sign Language communications) of the communication channel and translate the communications to text. The machine-learning model may be configured to process the text to add punctuation and other annotations to improve readability of the text. The text may be presented within an object of graphical user interface 312 as the visual transcript. In some examples, the visual transcript may be in the same language as spoken by the patient. A machine-learning model (e.g., such as a classifier, etc.) may be used to predict the language spoken by the patient the speech of the patient. If other user devices connected to the communication session communicate using other languages, then other machine-learning models may be used to translate those communications into text and translate the text into the language of the patient. For example, if the patient and the healthcare provider speak English and Spanish, respectively, the transcript may present the Spanish spoken by the healthcare provider as English within the visual transcript of post-session environment 300.

In some examples, post-session environment 300 may filter the audio channel of the recording. For example, post-session environment 300 may execute a machine-learning model (e.g., a recurrent neural network and/or other deep neural networks) configured to perform speaker diarization (e.g., partition an audio stream with multiple people into homogeneous segments associated with each speaker). The machine-learning model may receive audio input corresponding to the recorded communication session and filter out the audio from a particular user device, user, and/or other source of audio. The filtered-out audio may not be stored with the recording or in post-session environment 300. For example, audio received by the audio channel from the first user device (e.g., the patient) may be removed from the visual transcript, the recording, and/or other audio recording mechanisms associated with the communication session to comply with HIPPA, privacy policies, etc. In some instances, the first user device may request removal of any personal information, including audio communicated by the first user device over the audio channel of the communication session.

In some examples, post-session environment 300 may process visual features of the of the video channel of the communication session. For example, post-session environment 300 may use one or more machine-learning models to filter out confidential patient information to comply with HIPAA, privacy policies, user requests, etc. The one or more machine-learning models may filter out the portion of the audio channel received from the first user device (as previously described), such that the recording only includes the portion of the audio channel received from the second user device. In another example, the one or more machine-learning models may be usable to filter out test results, scan results (e.g., MRI, CT, etc.), and other patient-specific objects shared through the collaborative window. The one or more machine-learning models may retain generalized health information and objects (e.g., such as diagrams of body parts, etc.), general treatment plans, diagnosis information, symptom analysis, etc.

In some examples, post-session environment 300 may provide information pertaining to one or more clinical trials that may be applicable to the first user device. The information may be provided via pop-up, digital brochure, digital pamphlet, video clip, any combination thereof, or the like. The first user device may be prompted to input contact information to receive additional information pertaining to a clinical trial of the one or more clinical trials. For example, if a patient is suffering from a knee injury, the patient may receive information about one or more experimental drugs, surgeries, and/or treatments applicable to the knee injury.

In some examples, the abstracted version of the recording (that excludes all personal identifiable information associated with the first user device) may be stored by the communication network along with abstracted versions of other recordings in a repository accessible to users of the communication network. The repository can be searched using the keywords of the recording as well labels generated by the communication network. The labels may be generated based on one or more overall contexts of the recording (e.g., the purpose of the communication session, the most frequently occurring keyword, reported symptoms, diagnosis, treatments, etc.). The online repository may be accessed through post-session environment 300, the Internet, the communication network, an environment of the communication network accessible to healthcare providers, any combination thereof, or the like. The online repository may be searchable via keyword, label, dates, ailment or diagnosis, healthcare provider, any combination thereof, or the like. For example, the first user device may search the repository for recordings of communication session regarding torn Anterior Cruciate Ligament (ACL). Healthcare providers may search the repository to identify sessions involving similar symptoms, diagnosis, treatments, patients, etc.

In some examples, the first user device may upload additional documentation associated with the recording. For example, the first user device may upload a food journal, test results, report from a specialist, images, videos, scans, notes, questions, any combination thereof, or the like. The second user device may receive a notification indicating the first user device has uploaded additional documentation. The second user device may review the additional documentation and transmit a message to the first user device, if necessary.

In some examples, the telehealth environment may include cryptographic watermarking (e.g., spatial watermarking, frequency watermarking, etc.) usable to authenticate a communication session, the users connected to the communication session (e.g., a patient, a healthcare provider, etc.), and/or the like. Watermarks may be inserted into the video and/or audio component of a communication session during the communication session (e.g., in real time at various times during the communication session or continuously throughout the communication session) and/or after the communication session (e.g., into the digital recording of the communication session as previously described). In some instances, the watermark may be inserted by the user device (e.g., user device 108, user device 112, etc.) before the video and/or audio of that user device is transmitted to the other devices connected to the communication session and/or to the communication network facilitating the communication session (e.g., communication network 120 of FIG. 1). In some instances, the watermark may be inserted by the communication network before transmitting the watermarked video and/or audio to the other devices connected to the communication session. For instance, the communication network may facilitate a communication session by receiving video and/or audio from each device connected to the communication session, processing the video and/or audio, and transmitting the processed video and/or audio to the other devices connected to the communication session. The communication network may insert the watermark into the video and/or audio during processing. In those instances, during the communication session, each device connected to the communication session may receive watermarked video and/or audio where the watermark is inserted in real time without impacting the performance of the communication session (e.g., introducing lag, visual impairments, audible impairments, etc.).

Watermarks may be inserted into any portion of the video and/or audio. In some instances, watermarks may be inserted into a portion of the video and/or audio that is less likely to be subject to tampering or watermark-defeating strategies. For instance, some watermarking techniques insert watermarks into a portion of a video frame that less likely to be visible (e.g., top one or more rows of the video frame, bottom one or more rows of the video frame, left most one or more columns of the video frame, etc.). But these watermarks can be easily identified and removed by cropping the video, which would remove the watermark. To prevent the unauthorized removal or tampering of watermarks, the watermarks may be inserted in a location of the video in which the watermarks are less likely to be detected or a location of the video which is less likely to be altered. For example, watermarks may be inserted by altering the pixels corresponding to a face of a user device, pixels corresponding to another portion of the user (hair, neck, arms, torso, etc.), pixels near a center of the video frame, etc. In some instances, the watermarks may be inserted into a different location within the video frame each time the watermark is to be inserted during the communication session to prevent unauthorized detection of the watermark and to prevent removal of every instance of the watermark. For example, a cryptographic watermark may be inserted into a video stream with a representation of a healthcare provider's face (e.g., camera view 224 as described in FIG. 2A and FIG. 2B) by modifying one or more contiguous pixels corresponding to the healthcare provider's face.

The cryptographic watermarking may be facilitated by a machine-learning model trained to incorporate cryptographic watermarking into a video stream (e.g., deep neural networks, convolutional neural networks, etc.). The machine-learning model may be trained to receive video or individual video frames, perform image segmentation to identify the portion of the video frame that corresponds to a face, and modify the video or video frame by modifying one or more pixels of the portion of the video frame corresponding to the face. The pixels may be modified by causing a first modification to the luminance or chrominance of the pixel to represent a first symbol and causing a second modification to the luminance or chrominance of the pixel to represent a second symbol. Each pixel of the watermark may be modified according to the first modification or the second modification such that the pixels of the watermark represent a sequence of symbols.

The cryptographical watermark may include a sequence of symbols that represent one or more characteristics of the communication session such as an identification of the healthcare provider, characteristics of the healthcare provider (e.g., current location, IP address, MAC address, profile of the user device operated by the healthcare provider, etc.), an identification of the patient, characteristics of the patient (e.g., current location, IP address, MAC address, profile of the user device operated by the patient, etc.), an identification of the communication session (e.g., an identifier, etc. that uniquely identifies a particular communication session, an identification other users or user devices connected to the communication session, a timestamp of when the watermark is inserted into the communication session, a timestamp corresponding to the start of the communication session, a time interval corresponding to the length of the communication session (for watermarks inserted after the communication session terminates), or any other information associated with a user or user device connected to the communication session or the communication session itself. The one or more characteristics may be encrypted (e.g., hashed, etc.) and converted into a sequence of symbols and passed to the machine-learning model (or a non-machine-learning) component that will insert the sequence of symbols into a video frame by modulating the luminance and/or chrominance of set of pixels of the video frame as previously described.

In some examples, the cryptographic watermarking may be incorporated according to a sampling rate associated with the video stream, the communication session, the telehealth environment, a communication network associated with the telehealth environment, etc. For example, the sampling rate may be 30 fps (frames-per-second), 60 fps, 120 fps, or any other sampling rate associated with video processing. The sampling rate may indicate how frequently watermarks are to be inserted into the communication session (e.g., every 30 frames, every 60 frames, every 120 frames, etc.). For example, if the sampling rate is 30 fps, then thirty frames per second would include the cryptographic watermark.

The cryptographic watermarking may be incorporated prior to the display of the associated video stream on the one or more user devices. The cryptographic watermarking may be incorporated in real-time and/or near-real-time. For example, a communication network may receive video input from a first user device of the one or more user devices, incorporate cryptographic watermarking to generate a modified video input, then present the modified video input to a second user device. The cryptographic watermarking may be incorporated at a central server and/or central controller associated with the communication network (e.g., such as communication network 120). Alternatively, or additionally, a user device of the one or more user devices may incorporate the cryptographic watermarking. For example, the first user device may generate a video output, incorporate cryptographic watermarking to generate a modified video output, transmit the modified video output to the communication system, and the communication network may present the modified video output to the second user device. In some examples, the cryptographic watermarking may be incorporated at a recipient user device of the one or more user devices. For example, the first user device may generate a video output, transmit the video output to the communication system, the communication network may transmit the video output to the second user device, the second user device may incorporate cryptographic watermarking to generate a modified video output, and the modified video input may be presented via the second user device.

A digital recording of the communication session may be generated for training machine-learning models, reference by a patient or a healthcare provider (or other party), for storage in a database, etc. In some examples, cryptographic watermarking may be incorporated into the recording of the communication session prior to storage and/or distribution of the recording (e.g., prior to access in a post-session environment as described in FIG. 3A and/or FIG. 3B). The cryptographic watermarking may be incorporated at a user device associated with the communication session and/or at a central server and/or central controller associated with the communication system.

In some instances, the cryptographic watermarking may be usable to define a chain of custody for a particular communication session and/or digital recording of a communication session. In addition to or in place of the cryptographic watermarking performed during the communication session, updated cryptographic watermarking may be implemented each time the digital recording of a communication session is accessed. For example, when a digital recording is accessed, the communication network may extract the sequence of symbols of the current cryptographic watermark (if present), decrypt the sequence of symbols can be decrypted into the one or more characteristics, append one or more additional characteristics to the one or more characteristics forming a set of characteristics, encrypt the set of characteristics forming a new sequence of symbols, and generating a new cryptographic watermark by encode the sequence of symbols into a video frame by modulating pixels of the video frame. The one or more additional characteristics may include a timestamp corresponding when the digital recording is accessed, an identification of a user that accessed the digital record, an identification of a device that accessed the recording, characteristics of the device that accessed the recording (e.g., IP address, MAC address, an identification of hardware and/or software installed thereon, etc.), an identification of a location of the device that accessed the recording, an identification of a duration of time in which the digital record is accessed, an identification of a portion of the digital recording accessed (e.g., if the digital record is not accessed uniformly), and/or the like. The updated cryptographic watermarking may be implemented by the communication network and/or the device that accesses the digital recording. The updated cryptographic watermarking may be implemented when the digital recording is first accessed or when terminating access to the digital recording (e.g., when the device accessing the digital recording no longer has access to the digital record). In some instances, instead of implementing the updated cryptographic watermarking when the digital recording is accessed, the communication network and/or the device accessing the digital record may implement parallel cryptographic watermarking by inserting cryptographic watermarks (according any of the previously described means) within a different portion of the video frames of the digital recording such that the digital recording may include two or more cryptographic watermarks.

By implementing updated cryptographic watermarking (or parallel cryptographic watermarking), the communication network may generate an auditable trail for each digital recording that identifies the users and devices that access a digital recording. The communication network may store a representation of the set of characteristics encoded into cryptographic watermarking as metadata associated with the digital recording. The updated cryptographic watermarking (or parallel cryptographic watermarking) may be compared to the metadata as another security measure to detect data corruption and/or data manipulation. If the metadata does not match the cryptographic watermarking, the digital recording may be flagged and prevented from further access until an audit can be performed.

In some examples, the telehealth environment may present live data visualization within the communication session. The live data visualization may be based on patient data including, but not limited to, data from a patient's electronic health record (EHR), data provided by or selected by a healthcare provider, data provided by the patient, and/or the like. The live data visualization can be activated during the communication sessions to create dynamic charts, graphs, spreadsheets, tables, infographics, video, etc. using the patient data. For example, a user device associated healthcare provider may generate a graphic to present within a collaborative window (e.g., collaborative window 204 as described in FIG. 2A). In some examples, the graphic may be pre-generated prior to the communication session (e.g., the healthcare provider downloads the graphic for access during the communication session). In some other examples, the graphic may be generated in real-time during the communication session. For example, the user device associated with the healthcare provider may present a graphic associated with a patient's A1C using data from the patient's health records. The graphic may be generated by the user device associated with the healthcare provider or by the communication network. In some examples, the graphic may be generated by a machine-learning model trained to generate visual representations of data (e.g., neural network, transformer, support vector machine, perceptron, k-nearest neighbors, random forest, Naïve Bayes, generative adversarial network, regression-based modeling such as linear or logistic, etc.).

The graphic may be augmented with additional data values such as inferences derived from the patient data, projections, trends, comparison with average patients or similar patients, etc. For example, the graphic can be augmented by interpolation (e.g., adding additional data values), regression analysis (e.g., trends, predict future data values, etc.), modeling, inferencing (e.g., based on values output from a machine-learning model as previously described or values output from a non-machine-learning-based algorithm). The augmented data may enable generation of graphics that include simulated data such as (but not limited to) interpolated or extrapolated data values. The augmented data may be used to represent predicted data values within a graphic. For example, a graphic representing a patient's weight may be augmented by extrapolating a hypothetical future weight for the patient if the patient does not incorporate one or more lifestyle changes. The augmented data and the representation of augmented with patient data within a graphic may controlled by a patient or healthcare provider to represent any type of trend, prediction, target values, minimum and/or maximum values for particular data value and/or a particular patient, etc. For example, the augmented data may be used to generate graphic to demonstrate regulation of a patient's A1C if a certain diabetes treatment is incorporated into the patient's treatment plan (e.g., such as a particular therapeutic, drug, etc.). The augmented data may be generated dynamically (e.g., a runtime during the communication session) using one or more machine-learning models or statistical algorithms (e.g., such as, but not limited to, neural network, transformer, support vector machine, perceptron, k-nearest neighbors, random forest, Naïve Bayes, generative adversarial network, regression-based modeling such as linear or logistic, etc.). The one or more machine-learning models may generate the augmented data by incorporating the patient's health record, the health records of additional patients (e.g., obfuscated to prevent unauthorized access, display, or distribution of patient records), published studies, survey results, drug data, publicly available health data, any combination thereof, or the like, into the calculation.

In some examples, biometric data associated with a patient may be received (and/or derived) before, during, and/or after a communication session. Biometric data may be received from sensors associated with the user device of the patient and/or the patient. Examples of sensors from which biometric data may be received include, but are not limited to, pulse oximeter (e.g., including pulse, oxygen saturation, etc.), electroencephalogram, electrocardiogram, thermometer, motion sensors (e.g., pedometer, accelerometer, gyroscope), sweat sensors, combinations thereof, or the like. Biometric data may be derived from other sources. For instance, biometric data such as temperature, pulse, blood pressure, body mass index, mood, pain, etc. can be detected using a images or video of the patient and/or the audio segments (e.g., by comparing vocal characteristics to baseline vocal characteristics, etc.). For example, a camera associated with a user device of the patient may receive a video stream of the patient's face and biometric data can be derived by comparing successive video frames of the video stream (e.g., pulse, blood pressure, body mass index, etc.).

In some examples, the telehealth environment may include one or more dictionaries that may be associated with a communication session. The one or more dictionaries may be selected by one or more user devices connected to the communication session. The one or more dictionaries may include one or more words, phrases, slang terms, terms of art, definitions, dialects, regional vocabularies, any combination thereof, or the like, that may be used over the duration of the communication session. The one or more dictionaries may also be selected according to a topic of discussion associated with the communication session. For example, if the patient indicated that she was requesting the communication session to seek treatment for her diabetes, then the one or more dictionaries may include words, phrases, definitions, etc. associated with diabetes and diabetes treatments (e.g., insulin, A1C, glucose, ketoacidosis, etc.). In some examples, the one or more dictionaries may be selected according to a language spoken by a user of a user device associated with the communication session. For example, a user associated with the communication session may speak a particular regional dialect of Spanish and may therefore select to include a dictionary associated with the particular regional dialect.

The one or more dictionaries may be selected in advance (e.g., based on an anticipated topic of discussion, etc.) and/or selected in real time based on a of words or phrases spoken by the patient or the healthcare provider. For example, the one or more dictionaries may be selected manually by one or more user devices associated with the communication session. Alternatively, or additionally, a machine-learning model (e.g., a topic classifier model such as a transformer, etc.) may select the one or more dictionaries based on words or phrase spoken during the communication session, a topic of discussion, patient data, a location of the user device associated with the patient, combinations thereof, or the like. Alternatively, or additionally, the one or more dictionaries may be selected by keyword matching words, phrases, or topics spoken by the healthcare provider or patient. The one or more dictionaries may be referenced during generation of a transcript of the communication session (e.g., generated in real time during the communication session or after the communication session) to augment the transcript. The augmented transcript may include links or hyperlinks for words or phrases referenced in the one or more dictionaries. When a word or phrase in the transcript is selected or hovered over (e.g., with a cursor, a finger-gesture for touchscreen interfaces, etc.), the link may execute to display the corresponding definition of the selected word or phrase from the one or more dictionaries.

The one or more dictionaries may be generated based on words or phrases associated with a particular ailment, disease, condition, treatment, drug, therapeutic, medical specialty, topic, etc. In some instances, a large language model (such as a transformer-based model) or other machine-learning model may be used to generate a dictionary associated with a particular topic. The healthcare provider and/or the patient may modify the dictionary by adding additional words or phrases, adding slang or dialect-specific versions of words or phrases, adding definitions, removing words or phrases, modifying definitions, removing definitions, removing slang and/or dialect-specific version of words or phrases, adding or removing topics, modifying topic descriptions, etc. The modified dictionary may be reprocessed by the large language model to add, remove, and/or modify data of the dictionary (e.g., words, phrases, definitions, etc.) based on the modification introduced the healthcare provider and/or the patient. Reprocessing the dictionary using the large language model may also operate as a reinforcement learning iteration that may update the large language model (e.g., adjusting weights, etc.). The reinforcement iteration may improve the quality and/or accuracy of subsequent dictionaries generated by the large language model.

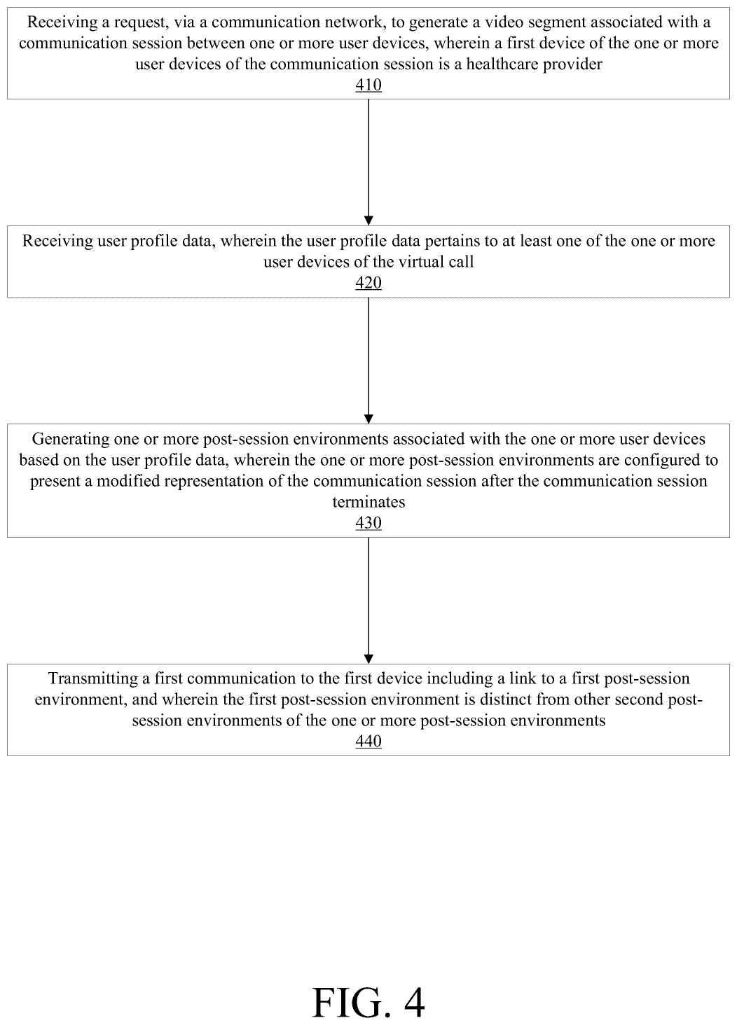

FIG. 4 illustrates a flowchart of an example process for generating a post-session environment of the communication network configured for a particular user device according to aspects of the present disclosure. At block 410, the computing device may receive a request to establish a communication session between one or more user devices. The computing device may be a component (e.g., a processing node, device, etc.) of a communication network (e.g., such as communication network 120 of FIG. 1, or the like). A first user device of the one or more user devices may be operated by a patient and a second user device of the one or more user devices may be operated by a healthcare provider.

In some examples, the first user device may transmit a communication session request to the computing device to request a communication session including the first user device and the second user device. The communication session may be facilitated over one or more communication channels (e.g., telephone or other audio-based communication channels, video or other audiovisual-based communication session, text such as text messaging or instant messaging, combinations thereof, or the like). In some instances, the communication channel may be asynchronous, or communications may be transmitted over the communication channel asynchronously. For example, the first user device may communicate over an audio channel and lack a capability of receiving or processing data received over a video channel (e.g., due to permissions or other security settings, processing capabilities, available resources, available bandwidth, etc.). The second user device may communicate over both audio and video, etc. such that the first user device may receive audio from the second user device and the second user device may receive both audio and video from the first user device. In other examples, the second user device may transmit a communication session request to the computing device to request a communication session including the first user device and the second user device.

The communication session request may include a set of communication session parameters usable to configure a communication session. The set of communication session parameters may include, but are not limited to, a quantity of user devices that are authorized to connect to the communication session, an identification of the user devices or the users thereof (e.g., such a device identifier, Internet Protocol address, email address, phone number, username, a user identifier, combinations thereof, or the like), a length of the communication session, an identification of one or more communication channels authorized for the communication session (e.g., such as, audio, video, audiovisual, text messaging, email, instant messaging, combinations thereof, or the like), video settings, sound settings, collaborative environment parameters, privacy and/or encryption parameters, artificial intelligence accessibility, date and time, etc. The set of communication session parameters may be modified at any time prior to and/or during the communication session.

The computing device may transmit a notification to the one or more user devices invited to the communication session using information of the set of communication session parameters. The notification may include a representation of one or more communication session parameters of the set of communication session parameters. The computing device may receive a response from the one or more user devices (or user devices associated with the communication request) indicating that a user device accepts the communication session, declines the communication session, or request a modification to the communication session (with an identification of one or more alternative parameters corresponding to the requested modification).

At block 420, the computing device may receive user profile data, wherein the user profile data pertains to at least one of the one or more user devices of the communication session. User devices connected to the communication session may be associated with individual user profiles stored within the computing device. The individual user profiles may be generated and/or stored prior to the communication session. The user profile data within the individual user profiles may include, but is not limited to, demographic data (e.g., race, gender, ethnicity, understood languages, etc.), health and medical history (e.g., height, weight, past surgeries, etc.), preferences (e.g., preferred medications, preferred medication ingestion, preferred language or form of communication, etc.), allergies, familial status, contact information, insurance information, data associated with prior communication sessions connected to by the user device associated with the individual user profile, healthcare provider information (e.g., primary care physician, OB/GYN, physical therapist, etc.), medical practice history, lifestyle data (e.g., smoking and alcohol frequency, exercise frequency, tendency to remember to take daily medication, tendency to cancel/reschedule/no-show communication sessions and/or other healthcare appointments, stress level, pollution level of living environment, cleanliness, eating habits, etc.), any combination thereof, or the like. Data of the user profile may be usable to customize the communication session or features thereof to the at least one user device or the user thereof.

At block 430, the computing device may generate one or more post-session environments associated with the one or more user devices, wherein the one or more post-session environments are configured to present a modified representation of the communication session after the communication session terminates, and wherein the communication system is configured to generate a first post-session environment associated with the first user device and a second post-session environment associated with the second user device. The one or more post-session environments may contain any of the features and/or aspects described in FIGS. 3A-3B.

The post-session environments may contain a reconstruction of the communication session (e.g., containing audio, visual, etc. content outputted over the duration of the communication session, etc.), data derived from the communication session using one or more machine-learning models or users, combinations thereof, or the like. After the communication session terminates, the user devices connected to the communication session may receive a notification (e.g., via email, a telehealth environment notification, text message, instant message, push notification, direct message, etc.) indicating that an instance of a post-session environment has been generated for the user device. The post-session environment accessible to each user device may differ based on the user device. The post-session environment of each user device may present a representation of the communication session that was presented to that device during the communication session. The post-session environment generated for the first user device may include functions configured for patients (e.g., a chat interface to communicate with the healthcare provider or healthcare system, etc., information about a diagnosis provided by the healthcare provider during the communication session, information about a prescribed treatment, etc.). The post-session environment generated for the second user device may include functions configured for healthcare provider (e.g., medical records associated with the patient, notes generated during the communication sessions, treatments prescribed, ordered tests, referral information such as an identified specialist, etc.).

In some instances, the post-session environment may include additional data derived from the communication session such as additional information about objects presented in the user interface (including the collaborative window, etc.), statements made by the healthcare provider or patient, etc. The additional data may be generated automatically (e.g., by one or more of the machine-learning models previously described), by the healthcare provider, by the patient, or another entity that was connected to the communication session, combinations thereof, or the like.

At block 440, the computing device may transmit a communication to the first user device containing a link to the first post-session environment. The link may cause the first user device to establish a connect to the post-session environment defined for the first user device. In some instances, the link may be a hyperlink, a pointer (e.g., an address to a location in local or remote memory, etc.), a function that automatically executes to connect to the post-session environment, or the like. The link may be executed by the patient (e.g., such as by activating a hyperlink, etc.) or automatically by the first user devices. The computing device may transmit a second communication containing a second link to a second post-session environment defined for the second user device. The first post-session environment may be distinct from the second post-session environment. The communication and the second communication may be transmitted over any communication channel (e.g., email, text message, direct messaging, instant messaging, push notification, patient portal notification, phone call, computing device, any combination thereof, or the like). The first session environment and/or the second post-session environment may be established via a webpage, a patient portal, a telehealth environment, a virtualized environment (e.g., a virtual machine, container, etc.), an application associated with the computing device, any combination thereof, or the like.

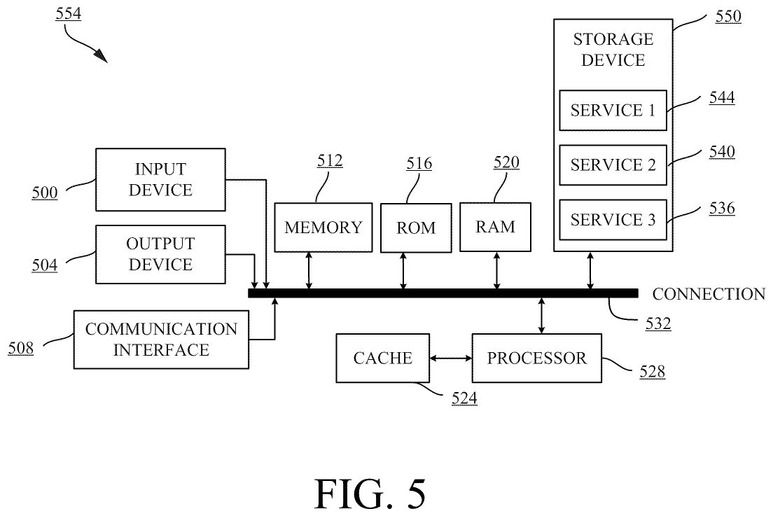

FIG. 5 illustrates an example computing device according to aspects of the present disclosure. For example, computing device 500 can implement any of the systems or methods described herein. In some instances, computing device 500 may be a component of or included within a media device. The components of computing device 500 are shown in electrical communication with each other using connection 506, such as a bus. The example computing device architecture 500 includes a processor (e.g., CPU, processor, or the like) 504 and connection 506 (e.g., such as a bus, or the like) that is configured to couple components of computing device 500 such as, but not limited to, memory 520, read only memory (ROM) 518, random access memory (RAM) 516, and/or storage device 508, to processing unit 510.

Computing device 500 can include a cache 502 of high-speed memory connected directly with, in close proximity to, or integrated within processor 504. Computing device 500 can copy data from memory 520 and/or storage device 508 to cache 502 for quicker access by processor 504. In this way, cache 502 may provide a performance boost that avoids delays while processor 504 waits for data. Alternatively, processor 504 may access data directly from memory 520, ROM 517, RAM 516, and/or storage device 508. Memory 520 can include multiple types of homogenous or heterogeneous memory (e.g., such as, but not limited to, magnetic, optical, solid-state, etc.).

Storage device 508 may include one or more non-transitory computer-readable media such as volatile and/or non-volatile memories. A non-transitory computer-readable medium can store instructions and/or data accessible by computing device 500. Non-transitory computer-readable media can include, but is not limited to magnetic cassettes, hard-disk drives (HDD), flash memory, solid state memory devices, digital versatile disks, cartridges, compact discs, random access memories (RAMs) 525, read only memory (ROM) 520, combinations thereof, or the like.

Storage device 508, may store one or more services, such as service 1 510, service 2 512, and service 3 514, that are executable by processor 504 and/or other electronic hardware. The one or more services include instructions executable by processor 504 to: perform operations such as any of the techniques, steps, processes, blocks, and/or operations described herein; control the operations of a device in communication with computing device 500; control the operations of processing unit 510 and/or any special-purpose processors; combinations therefor; or the like. Processor 504 may be a system on a chip (SOC) that includes one or more cores or processors, a bus, memories, clock, memory controller, cache, other processor components, and/or the like. A multi-core processor may be symmetric or asymmetric.

Computing device 500 may include one or more input devices 522 that may represent any number of input mechanisms, such as a microphone, a touch-sensitive screen for graphical input, keyboard, mouse, motion input, speech, media devices, sensors, combinations thereof, or the like. Computing device 500 may include one or more output devices 524 that output data to a user. Such output devices 524 may include, but are not limited to, a media device, projector, television, speakers, combinations thereof, or the like. In some instances, multimodal computing devices can enable a user to provide multiple types of input to communicate with computing device 500. Communications interface 526 may be configured to manage user input and computing device output. Communications interface 526 may also be configured to managing communications with remote devices (e.g., establishing connection, receiving/transmitting communications, etc.) over one or more communication protocols and/or over one or more communication media (e.g., wired, wireless, etc.).

Computing device 500 is not limited to the components as shown in FIG. 5. Computing device 500 may include other components not shown and/or components shown may be omitted.

As used below, any reference to a series of examples is to be understood as a reference to each of those examples disjunctively (e.g., “Examples 1-4” is to be understood as “Examples 1, 2, 3, or 4”).

Example 1 is a computer-implemented method, comprising: receiving a request, via a communication system, to generate a video segment associated with a communication session between one or more user devices, wherein a first device of the one or more user devices of the communication session is a healthcare provider; receiving user profile data, wherein the user profile data pertains to at least one of the one or more user devices of the communication session; generating one or more post-session environments associated with the one or more user devices based on the user profile data, wherein the one or more post-session environments are configured to present a modified representation of the communication session after the communication session terminates; and transmitting a first communication to the first device including a link to a first post-session environment, and wherein the first post-session environment is distinct from a second post-session environment of the one or more post-session environments.

Example 2 is the computer-implemented method of example(s) 1, further comprising: generating a temporary environment for at least one of the one or more user devices of the communication session, wherein the temporary environment includes at least one of a tutorial for the communication session, a symptom monitor, a data upload prompt, and a questionnaire.

Example 3 is the computer-implemented method of example(s) 1-2, wherein at least one of the one or more post-session environments is a captioned version of a reconstruction of the communication session, wherein the captioned version also contains a transcript of the communication session.

Example 4 is the computer-implemented method of example(s) 1-3, wherein at least one of the one or more user devices of the communication session is associated with more than one of the one or more post-session environments.

Example 5 is the computer-implemented method of example(s) 1-4, wherein at least one of the one or more post-session environments is a meta-tagged copy of a reconstruction of the communication session, whereby the meta-tagged copy may be searched by keywords or time stamps.

Example 6 is the computer-implemented method of example(s) 1-5, further comprising: storing the second post-session environment in a user library, wherein the user library contains one or more prior post-session environments pertaining to prior communication sessions, and wherein the user library is accessible to a second device.

Example 7 is the computer-implemented method of example(s) 1-6, wherein the communication session may be a bidirectional video call and wherein a recording of the communication session contains consolidated video content from the one or more user devices.

Example 8 is the computer-implemented method of example(s) 1-7, wherein the user profile data is generated by at least one of a machine learning algorithm, prior communication sessions, and data gathered prior to the communication session.

Example 9 is a system comprising of one or more processor and a non-transitory computer-readable medium storing instructions that when executed by the one or more processors cause the one or more processors to perform the methods of any of example(s) 1-8.

Example 10 is a non-transitory computer-readable medium storing instructions that when executed by the one or more processors cause the one or more processors to perform the methods of any of example(s) 1-8.

The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored in a form that excludes carrier waves and/or electronic signals. Examples of a non-transitory medium may include, but are not limited to, a magnetic disk or tape, optical storage media such as compact disk (CD) or digital versatile disk (DVD), flash memory, memory, or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

Some portions of this description describe examples in terms of algorithms and symbolic representations of operations on information. These operations, while described functionally, computationally, or logically, may be implemented by computer programs or equivalent electrical circuits, microcode, or the like. Furthermore, arrangements of operations may be referred to as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In some examples, a software module can be implemented with a computer-readable medium storing computer program code, which can be executed by a processor for performing any or all of the steps, operations, or processes described.

Some examples may relate to an apparatus or system for performing any or all of the steps, operations, or processes described. The apparatus or system may be specially constructed for the required purposes, and/or it may comprise a general-purpose computing device selectively activated or reconfigured by a computer program stored in memory of computing device. The memory may be or include a non-transitory, tangible computer readable storage medium, or any type of media suitable for storing electronic instructions, which may be coupled to a bus. Furthermore, any computing systems referred to in the specification may include a single processor or multiple processors.

While the present subject matter has been described in detail with respect to specific examples, it will be appreciated that those skilled in the art, upon attaining an understanding of the foregoing, may readily produce alterations to, variations of, and equivalents to such embodiments. Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter. Accordingly, the present disclosure has been presented for purposes of example rather than limitation, and does not preclude the inclusion of such modifications, variations, and/or additions to the present subject matter as would be readily apparent to one of ordinary skill in the art.

For clarity of explanation, in some instances the present disclosure may be presented as including individual functional blocks including functional blocks comprising devices, device components, steps or routines in a method embodied in software, or combinations of hardware and software. Additional functional blocks may be used other than those shown in the figures and/or described herein. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Individual examples may be described herein as a process or method which may be depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but may have additional steps not shown. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general-purpose computer, special purpose computer, or a processing device to perform a certain function or group of functions. Portions of computer resources used can be accessible over a network. The computer executable instructions may be, for example, binaries, intermediate format instructions such as assembly language, firmware, source code, etc.

Devices implementing the methods and systems described herein can include hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. The program code may be executed by a processor, which may include one or more processors, such as, but not limited to, one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A processor may be a microprocessor; conventional processor, controller, microcontroller, state machine, or the like. A processor may also be implemented as a combination of computing components (e.g., a combination of a DSP and a microprocessor, a plurality of microprocessors, one or more microprocessors in conjunction with a DSP core, or any other such configuration). Accordingly, the term “processor,” as used herein may refer to any of the foregoing structure, any combination of the foregoing structure, or any other structure or apparatus suitable for implementation of the techniques described herein. Functionality described herein also can be embodied in peripherals or add-in cards. Such functionality can also be implemented on a circuit board among different chips or different processes executing in a single device, by way of further example.

In the foregoing description, aspects of the disclosure are described with reference to specific examples thereof, but those skilled in the art will recognize that the disclosure is not limited thereto. Thus, while illustrative examples of the disclosure have been described in detail herein, it is to be understood that the inventive concepts may be otherwise variously embodied and employed, and that the appended claims are intended to be construed to include such variations. Various features and aspects of the above-described disclosure may be used individually or in any combination. Further, examples can be utilized in any number of environments and applications beyond those described herein without departing from the broader spirit and scope of the disclosure. The disclosure and figures are, accordingly, to be regarded as illustrative rather than restrictive.

The various illustrative logical blocks, modules, circuits, and algorithm steps described in connection with the embodiments disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or media devices of the computing platform. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for case of explanation only and are not meant to be limiting.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

1. A computer-implemented method, comprising:
receiving a request, via a communication system, to generate a video segment associated with a communication session between one or more user devices, wherein a first device of the one or more user devices of the communication session is a healthcare provider;
receiving user profile data, wherein the user profile data pertains to at least one of the one or more user devices of the communication session;
generating one or more post-session environments associated with the one or more user devices based on the user profile data, wherein the one or more post-session environments are configured to present a modified representation of the communication session after the communication session terminates; and
transmitting a first communication to the first device including a link to a first post-session environment, and wherein the first post-session environment is distinct from a second post-session environment of the one or more post-session environments.

2. The computer-implemented method of claim 1, further comprising:
generating a temporary environment for at least one of the one or more user devices of the communication session, wherein the temporary environment includes at least one of a tutorial for the communication session, a symptom monitor, a data upload prompt, and a questionnaire.

3. The computer-implemented method of claim 1, wherein at least one of the one or more post-session environments is a captioned version of a reconstruction of the communication session, wherein the captioned version also contains a transcript of the communication session.

4. The computer-implemented method of claim 1, wherein at least one of the one or more user devices of the communication session is associated with more than one of the one or more post-session environments.

5. The computer-implemented method of claim 1, wherein at least one of the one or more post-session environments is a meta-tagged copy of a reconstruction of the communication session, whereby the meta-tagged copy may be searched by keywords or time stamps.

6. The computer-implemented method of claim 1, further comprising:
storing the second post-session environment in a user library, wherein the user library contains one or more prior post-session environments pertaining to prior communication sessions, and wherein the user library is accessible to a second device.

7. The computer-implemented method of claim 1, wherein the communication session may be a bidirectional video call and wherein a recording of the communication session contains consolidated video content from the one or more user devices.

8. The computer-implemented method of claim 1, wherein the user profile data is generated by at least one of a machine learning algorithm, prior communication sessions, and data gathered prior to the communication session.

9. A system comprising:
one or more processors and a non-transitory computer-readable medium storing instructions that when executed by the one or more processors cause the one or more processors to perform operations that include:
receiving a request, via a communication system, to generate a video segment associated with a communication session between one or more user devices, wherein a first device of the one or more user devices of the communication session is a healthcare provider;
receiving user profile data, wherein the user profile data pertains to at least one of the one or more user devices of the communication session;
generating one or more post-session environments associated with the one or more user devices based on the user profile data, wherein the one or more post-session environments are configured to present a modified representation of the communication session after the communication session terminates; and
transmitting a first communication to the first device including a link to a first post-session environment, and wherein the first post-session environment is distinct from a second post-session environment of the one or more post-session environments.

10. The system of claim 9, further comprising:
generating a temporary environment for at least one of the one or more user devices of the communication session, wherein the temporary environment includes at least one of a tutorial for the communication session, a symptom monitor, a data upload prompt, and a questionnaire.

11. The system of claim 9, wherein at least one of the one or more post-session environments is a captioned version of a reconstruction of the communication session, wherein the captioned version also contains a transcript of the communication session.

12. The system of claim 9, wherein at least one of the one or more user devices of the communication session is associated with more than one of the one or more post-session environments.

13. The system of claim 9, wherein at least one of the one or more post-session environments is a meta-tagged copy of a reconstruction of the communication session, whereby the meta-tagged copy may be searched by keywords or time stamps.

14. The system of claim 9, further comprising:
storing the second post-session environment in a user library, wherein the user library contains one or more prior post-session environments pertaining to prior communication sessions, and wherein the user library is accessible to a second device.

15. The system of claim 9, wherein the communication session may be a bidirectional video call and wherein a recording of the communication session contains consolidated video content from the one or more user devices.

16. The system of claim 9, wherein the user profile data is generated by at least one of a machine learning algorithm, prior communication sessions, and data gathered prior to the communication session.

17. A non-transitory computer-readable medium storing instructions that when executed by one or more processors cause the one or more processors to perform operations that include:
receiving a request, via a communication system, to generate a video segment associated with a communication session between one or more user devices, wherein a first device of the one or more user devices of the communication session is a healthcare provider;
receiving user profile data, wherein the user profile data pertains to at least one of the one or more user devices of the communication session;
generating one or more post-session environments associated with the one or more user devices based on the user profile data, wherein the one or more post-session environments are configured to present a modified representation of the communication session after the communication session terminates; and
transmitting a first communication to the first device including a link to a first post-session environment, and wherein the first post-session environment is distinct from a second post-session environment of the one or more post-session environments.

18. The non-transitory computer-readable medium of claim 17, the operations further comprising:
generating a temporary environment for at least one of the one or more user devices of the communication session, wherein the temporary environment includes at least one of a tutorial for the communication session, a symptom monitor, a data upload prompt, and a questionnaire.

19. The non-transitory computer-readable medium of claim 17, wherein at least one of the one or more post-session environments is a captioned version of a reconstruction of the communication session, wherein the captioned version also contains a transcript of the communication session.

20. The non-transitory computer-readable medium of claim 17, wherein at least one of the one or more user devices of the communication session is associated with more than one of the one or more post-session environments.
